(19) United States
(12) Patent Application Publication
(10) Pub. No.: US 2008/0069800 A1
(43) Pub. Date: Mar. 20, 2008

(54) System for High Production of Natural and Personalized Interferons

(75) Inventor: Luwen Zhang, Lincoln, NE (US)

Correspondence Address: SHERIDAN ROSS PC, 1560 BROADWAY, SUITE 1200, DENVER, CO, 80202, US

(73) Assignee: BOARD OF REGENTS OF THE UNIVERSITY OF NEBRASKA, Lincoln, NE (US)

(21) Appl. No.: 11/748,161

(22) Filed: May 14, 2007

Related U.S. Application Data
(63) Continuation of application No. PCT/US07/68265, filed on May 4, 2007.
(60) Provisional application No. 60/797,915, filed on May 4, 2006.

(51) Int. Cl. A61K 38/21; A61P 31/12; C07K 14/555; C12P 21/00

(52) U.S. Cl. 424/85.4; 435/69.51; 530/351

(57) ABSTRACT

The present invention generally relates to systems and methods for the production of high amounts of personalized interferons (IFNs), which are IFNs produced from the cells of the patient to whom the IFN is to be administered, for therapeutic and research uses. The system can also be used to produce high amounts of natural IFNs.

### CROSS-REFERENCE TO RELATED APPLICATIONS

This application claims the benefit of priority under 35 U.S.C. § 120 of International Application No. PCT/US07/68265, filed May 4, 2007, which claims the benefit of priority of U.S. Provisional Application No. 60/797,915,filed May 1, 2006. The entire disclosure of each application is hereby incorporated by reference.

### GOVERNMENT SUPPORT

This invention was supported, in part, by federally funded Grant Nos. AI059132 and CA108951, each awarded by the National Institutes of Health. The government has certain rights to this invention.

### BACKGROUND OF THE INVENTION

Epstein-Barr virus (EBV) is a ubiquitous human gamma herpesvirus virus and more than 90% of adults are EBV carriers. EBV is able to persist in the serological positive immune host for life. Although harmless in majority of the population, EBV has oncogenic potential and is associated with the development of several human diseases, including nasopharyngeal carcinoma (NPC) and Burkitt's lymphoma (BL) (34, 54). In in vitro tissue culture systems, EBV can efficiently infect and transform resting human B lymphocytes into lymphoblastoid cell lines (LCLs) (34, 54). These LCLs are immortalized human lines and can grow indefinitely.

The biologic hallmark of EBV-cell interaction is latency. Three types of latency have been described, each having its own distinct pattern of gene expression. Type I latency is exemplified by BL tumors in vivo. EBV nuclear antigen 1 (EBNA-1) protein is expressed in this form of latency. Type II latency is exemplifed by NPC and Hodgkin's disease. EBNA-1, latent membrane protein 1 (LMP-1), LMP-2A and LMP-2B proteins, are expressed in type II latency. Type III latency is represented by lymphoblastoid cell lines (LCLs). Nine viral proteins are expressed in type III latency, including six nuclear proteins (EBNA-1, EBNA-2, EBNA-3A, EBNA-3B, EBNA-3C and EBNA-LP) and three integral membrane proteins (LMP-1, LMP-2A and LMP-2B) (reviewed in (34, 54)).

Interferons (IFNs) are a group of small proteins made by the body in response to viral infections. The body produces different types and amounts of interferon to fight different types of infection. IFNs have been widely used in therapeutic approaches to treat booth Hepatitis C virus (HCV) infection and Hepatitis B virus (HBV) infection, and IFNs are also used therapeutically to stop the growth and spread of cancer cells (e.g., kidney, malignant melanoma, multiple myeloma, carcinoid tumors, Karposi's sarcoma, and some types of lymphoma and leukemia) and to treat diseases such as multiple sclerosis (MS), and other diseases of viral, malignant, angiogenic, allergic, inflammatory, and fibrotic origin.

Type I interferons (α, β, θ, ω) are a family of products related to genes situated on chromosome 9 in the human. The biological activities of these proteins are wide ranging, and include virus inhibition, reduced tumor proliferation, antigen modulation, and immunomodulation (56, 57). Type II interferon (γ), in addition to having a pivotal role in host defense, has been associated under some conditions with the pathogenesis of chronic inflammatory and autoimmune disease. The α- and β-interferons (IFN-α and IFN-β, respectively) have demonstrated the greatest medical usefulness as therapeutics to date.

There are a large number of type I IFN genes in the human: 13 IFN-α genes, 1 IFN-β gene, and 1 IFN-ω gene (55). However, a unique role for IFN-β for a fully effective antiviral response, which cannot be compensated for by IFN-α, has been documented (17).

Currently, the production of natural IFNs for medical use is primarily achieved by infection of human cells by virus, followed by purification of the IFN from the cell. The cell sources are typically cultured cell lines or primary peripheral blood leukocytes (PBMCs) isolated from fresh blood. IFNs are also generated using recombinant technology by fermentation of genetically engineered bacteria. Although improved significantly, current IFN regimens still have at least two serious problems. The first problem is low efficiency: IFN is a treatment for hepatitis C and melanoma patients, yet only 50% of hepatitis C and 10-15% melanoma patients are responsive to current IFN treatments. The second problem is the presence of side effects: IFNs have been characterized by a number of side effects in patients from flu-like symptoms to organ failure. In addition, these side effects often hamper the attainment of optimal dose intensity and sometimes even necessitate premature cessation of therapy. For many patients, however, there is no alternative for therapy with IFN-α.

Given the great demand for IFNs for use in the treatment of diseases ranging from viral infection, to tumors, to autoimmune disease, there is a continued need in the art for the provision of improved systems and methods of efficiently producing large quantities of IFNs that are highly effective, safer and well-tolerated by the patient.

### SUMMARY OF THE INVENTION

The present invention generally relates to systems and methods for the production of high amounts of personalized interferons (IFNs), i.e., IFNs are produced from the cells of the patient to whom the IFNs are to be administered (FIG. 1), for therapeutic and research uses. The system can also be used to produce high amounts of natural IFNs.

In one embodiment, the invention provides a method to produce interferon (IFN), comprising providing a cell that has been primed for interferon production by infection of the cell with a herpesvirus or transfection of the cell with a portion of a herpesvirus genome, infecting the cell with a virus or providing the cell with a virus-like stimulus to induce the cell to produce IFN, and recovering the IFN from the cell.

In some embodiments, the cell is a human cell, a peripheral blood mononuclear cell, a lymphocyte or a B lymphocyte.

In certain embodiments, the herpesvirus is a gamma herpesvirus such as Epstein-Barr virus (EBV).

In some embodiments, the portion of the herpesvirus genome comprises a nucleic acid molecule encoding LMP-1. In other embodiments, the portion of the herpesvirus genome further comprises at least one nucleic acid molecule encoding a protein selected from: EBNA-1, EBNA-2, EBNA-3A, EBNA-3B, EBNA-3C and EBNA-LP, LMP-2A and LMP-2B. In yet another embodiment, the portion of the herpesvirus genome comprises a nucleic acid molecule encoding at least one CTAR domain of LMP-1.

In certain embodiments, the virus is selected from the group consisting of: Sendai virus, Newcastle disease virus and vesicular stomatitis virus. In some embodiments, the virus is Sendai virus. In other embodiments, the virus-like stimulus is double-stranded RNA.

In additional embodiments, the step of recovering IFN from the cell comprises isolating IFN from the cell or a lysate thereof. In other embodiments, the stop of recovering IFN from the cell comprises purifying IFN from the cell or a lysate thereof.

In some embodiments, the IFN is a type I interferon, interferon-α or a species thereof, or interferon-β or a species thereof.

In certain embodiments, the cell is isolated from a human subject. In some embodiments, the step of recovering the IFN from the cell isolated from a human subject comprises purifying IFN from the cell or a lysate thereof. In yet other embodiments, method comprises administering the purified IFN to the human subject from which the cell was isolated. In certain embodiments, the subject has a disease or condition selected from the group consisting of: Hepatitis C virus (HCV) infection, Heptatis B virus (HBV) infection, cancer, and multiple sclerosis (MS).

In certain embodiments, the invention includes the isolated or purified IFN produced by the methods of the present invention.

In some embodiments, the invention provides a method to produce interferon (IFN), comprising providing a cell that expresses LMP-1 or a portion thereof that contains at least one CTAR domain, infecting the cell with a virus or providing the cell with a virus-like stimulus to induce the cell to produce IFN, and recovering the IFN from the cell. In some embodiments, the cell comprises an expression vector comprising a nucleic acid molecule encoding LMP-1 or a portion thereof that contains at least one CTAR domain.

In addition embodiments, the invention provides a method to produce personalized interferon (IFN) comprising obtaining a cell from a subject, priming the cell for interferon production by infection of the cell with a herpesvirus or transfection of the cell with a portion of a herpesvirus genome, infecting the cell with a virus to induce the cell to produce IFN, and recovering the IFN from the cell.

In further embodiments, the invention provides a method to produce personalized interferon (IFN) comprising obtaining a cell from a subject, transfecting LMP-1 or a portion thereof that contains at least one CTAR domain into the cell, infecting the cell with a virus to induce the cell to produce IFN, and recovering the IFN from the cell.

In some embodiments, the invention provides a method to treat a patient with a disease or condition capable of being treated by interferon (IFN) therapy comprising obtaining a cell from the patient, priming the cell for interferon production by infection of the cell with a herpesvirus or transfection of the cell with a portion of a herpesvirus genome, infecting the cell with a virus to induce the cell to produce IFN, isolating the IFN from the cell, and administering the isolated IFN to the patient. In certain embodiments, the patient has a disease or condition selected from the group consisting of: Hepatitis C virus (HCV) infection, Hepatitis B virus (HBV) infection, cancer, and multiple sclerosis (MS). In some embodiments, the patient has hepatitis.

In further embodiments, the invention provides a method to treat a patient with a disease or condition treatable with interferon (IFN) therapy comprising administering isolated IFN to the patient, wherein the isolated IFN has been produced by the process comprising infecting an herpesvirus-immortalized type III latency cell with a virus to induce the cell to produce IFN, and isolating the IFN from the cell. In certain embodiments, the step of isolating comprises purifying the IFN.

### BRIEF DESCRIPTION OF THE FIGURES

FIG. 1 is a representation of an example of a personalized IFN production system.

FIG. 2 is a demonstration of the generation of personalized IFNs. Panel A is a schematic diagram of the procedures that may be used. Panel B illustrates the concentrations of several IFN-α subtypes produced by LCLs generated from three individuals along with standard deviations. Panel C demonstrates the production of high levels of IFN-β from personalized cell lines infected by Sendai virus. Specific protections of IFN-β and GAPDH RNAs are indicated. In Panels B and C, the “+” and “−” symbols indicate with or without Sendai virus infection, respectively.

FIG. 3 demonstrates that EBV latency cells are primed to produce IFN-β upon infection. Panel A shows levels of IFN-β RNA upon infection with Sendai virus. Specific protections of IFN-β and GAPDH RNAs are indicated. Panel B shows the expression levels of LMP-1 in EBV latency cells. Lysates from BL41, BL41-EBV, SavI, and SavIII were used for western blot analysis with LMP-1 and tubulin antibodies. In both panels, the “+” and “−” symbols indicate with or without Sendai virus infection, respectively.

FIG. 4 shows that IFNs produced by infection of EBV latency cells are functional. Panel A demonstrates that IFNs are secreted into the culture media. Standard deviations are shown. The “+” and “−” symbols indicate with or without Sendai virus infection, respectively. Panel B shows that conditional media from EBV type III latency cells infected by Sendai virus induces STAT-1. The “+” and “−” symbols indicate with or without Sendai virus infection, respectively. Panel C demonstrates that IFN-α is involved in the induction of STAT-1. BL41 cells were treated with the conditional media (1:100. from BL41-EBV cells infected with Sendai), the neutralization antibody of IFN-α, and same amounts of normal rabbit serum (NRS) in various combinations as shown on the top. The expression of STAT-1 was examined 24 hours later. The identity of proteins is as shown.

FIG. 5 demonstrates that LMP-1 primes cells for IFN production. Panel A shows that LMP-1-expressing cells exhibit robust IFN production upon viral infection. DG75 cells were transfected with pcDNA3, LMP-1, or EBNA-2 expression plasmids. The transfected cells were isolated and equally split into two wells: one well of the cells was infected with Sendai virus for 6 hrs. Total RNA were isolated and used for RPA with IFN-β plus GAPDH probes. Yeast RNA was used as negative control. Specific protection of IFN-β and GAPDH RNAs are indicated. +/−: with or without virus infection. Panel B shows that IFN primes DG75 cells. DG75 cells were treated with IFN-α (200 IU/ml) overnight, and the cells were infected by Sendai virus for 6 hours. Total RNA was isolated and used for RPA. The “+” and “−” symbols indicate with or without Sendai virus infection, respectively.

FIG. 6 shows that signaling events from LMP-1 are required for robust IFN production. Panel A shows that cells expressing an LMP-1 mutant fail to produce IFN upon viral infection. DG75 cells were transfected with pcDNA3, LMP-1, or LMP-DM expression plasmids. The transfected cells were isolated and half of the cells were infected with Sendai virus for 6 hours. Total RNA was isolated and used for RPA with IFN-β plus GAPDH probes. Yeast RNA was used as negative control. Specific protections of IFN-β and GAPDH RNAs are indicated. The “+” and “−” symbols indicate with or without Sendai virus infection, respectively. Panel B shows the expression of target proteins in transfected cells. Lysates from transfected and enriched DG75 cells were used for western blot analysis with LMP-1 and tubulin antibodies. The identity of proteins is as shown.

FIG. 7 demonstrates that NF-κB is involved in LMP-1 mediated priming. Panel A shows that NF-κB is necessary for high IFN production. DG75 cells were transfected with pcDNA3, LMP-1, LMP-1+IκB, or NF-κB(p65+p50) expression plasmids. The transfected cells were enriched and infected with Sendai virus for 6 hours. IFN production was measured by RPA. The identity of RNAs is as shown. The “+” and “−” symbols indicate with or without Sendai virus infection, respectively. Panel B shows the expression of target proteins in the transfected cells by western blot analysis with LMP-1 and tubulin antibodies. The identity of proteins is as shown.

FIG. 8 shows the involvement of IRF-7 in LMP-1-medited priming. Panel A demonstrates that ectopic expression of IRF-7 primes cells for IFN production. DG75 cells were transfected with IRF-7 or its mutant IRF-7K92E expression plasmids. The transfected cells were enriched and infected with Sendai virus for 6 hours. IFN production was measured by RPA. The identity of RNAs is as shown. The “+” and “−” symbols indicate with or without Sendai virus infection, respectively. Panel B shows the expression of target proteins in transfected cells by western blot analysis with IRF-7 and tubulin antibodies. The identity of proteins is as shown.

FIG. 9 demonstrates that personalized IFNs are produced from EBV-transformed cells at similar levels as commercially available cell sources such as Namalwa and freshly isolated PBMCs. The “+” and “−” symbols indicate with or without Sendai virus infection, respectively.

### DESCRIPTION OF THE INVENTION

The present invention generally relates to systems and methods for the production of interferons (IFNs) for therapeutic and research uses. First, the systems and methods of the present invention produce high levels of “personalized” IFN, which is IFN produced from the cells of the patient to whom the IFN is to be administered, which greatly reduces the potential for natural antibody reactions against the IFN and therefore, improves circulation time and efficacy in the patient, and particularly, in “IFN-insensitive” or “IFN-nonresponsive” patients. The system and method of the present invention can provide a virtually unlimited amount of IFN produced from a patient's own cells. Second, the systems and methods of the invention produce natural IFN, which has advantages for in vivo use over recombinant IFN.

In one aspect, the method to produce IFN comprises priming a cell for interferon production by infection of the cell with a herpesvirus or transfection of the cell with a portion of a herpesvirus genome, infecting the cell with a virus to induce the cell to produce IFN, and recovering the IFN from the cell. The present inventors have discovered that herpesvirus infection of a cell followed by infection with a second virus greatly increases the amount of IFNs produced by the cell. In some embodiments, the cells are derived from an individual and the isolated IFN is personalized for the individual.

In another aspect, the present invention relates to the use of herpesvirus-transformed cells (e.g., EBV-transformed cells) that are induced to produce high titers of IFN. The cells are provided from an acceptable source (e.g., cell lines or freshly isolated cells, such as PBMCs), and in one embodiment, are isolated from the patient to whom the IFN is to be administered. The cells are then immortalized with a herpesvirus in vitro, and induced to produce IFN. The IFN can be isolated and purified from the cells, and then used in therapeutic applications or in other applications (e.g., research applications) as desired.

The present invention utilizes the ability of herpesvirus infection to prime cell for IFN production i order to produce significant quantities of natural IFN that can be “personalized,” if desired, by using cells from the patient to whom the IFN is to be administered. As used herein, “natural” IFN refers to IFN produced by cells capable of naturally producing IFN in response to various stimuli. Accordingly, natural IFNs are typically the same as the IFNs made by cells in vivo. In contrast, synthetic IFNs include IFNs produced using recombinant systems (e.g., expression of a recombinant IFN molecule in a bacterial system). Personalized IFNs may provide a significant advantage over current methods for production of natural IFN and further, enables production of a virtually unlimited supply of IFN from a person's own cells that would not be possible using primary cells alone. More particularly, primary cells isolated from an individual, such as primary B lymphocytes, would die relatively quickly in tissue culture, even though all nutrients and other factors are provided. However, herpesviruses such as EBV can be used to immortalize primary B cells, for example, from the blood and convert the B lymphocytes into cells that can be grown indefinitely in vitro. Thus, using the system and method of the present invention, and individual can have an unlimited source of his/her “personalized” cells available. Furthermore, these immortalized cells are type III latency cells, and are primed to produce high yields of IFNs upon superinfection.

In one aspect of the invention, cells primed for IFN production are infected to induce IFN production. As used herein, the terms “infection” or “infected” and “superinfection” or “superinfected” are used interchangeably. For example, a cell that has been previously infected with a virus such as EBV is sometimes referred to as “superinfected” when infected with a second virus such as Sendai. A cell that has been transfected with LMP-1, however, would be “infected” by exposure to Sendai virus. Each process involves essentially the same steps but with a different starting cell, and therefore the terms are used synonymously.

In one aspect of the invention, cells are primed for IFN production by infecting the cell will a herpesvirus such as EBV. The induction of IFN stimulated genes (ISGs) is a common phenomenon in herpesviruses, without the involvement of IFNs (7, 12, 46, 60, 81). Thus, other herpesviruses may be used in the present invention to induce a similar priming effect on IFN production. Herpesviruses suitable for use in the present invention include any member of the family Herpesviridae, such as alpha, beta and gamma herpesviruses. In certain embodiments the herpesvirus may be a human herpesvirus; in others, a herpesvirus capable of infecting a non-human mammal. Examples include bovine, equine, porcine, feline and canine herpesviruses. In some embodiments, the herpesvirus may be HHV-1, -2, -3, -4, -5, -6, -7, or -8. In one embodiment, the herpesvirus is EBV. In further embodiments, a portion of a herpesvirus genome sufficient to prime the cell for IFN production may be introduced into the cell (e.g., by infection or transfection). In one embodiment, the portion of a herpesvirus genome introduced into the cell comprises a gene or other nucleic acid molecule encoding LMP-1.

The present invention also relates to priming a cell for interferon production by transfection of the cell with a portion of a herpesvirus genome. The portion of a herpesvirus genome suitable for cell priming minimally comprises LMP-1, but may contain additional viral genes. In some embodiments, cells that express a portion of a herpesvirus genome containing LMP-1 may be used in place of cells that have been generated by infection with a herpesvirus. For example, many cell lines exist that express a portion of a herpesvirus genome including LMP-1 and each cell line may be infected with a virus such as Sendai to produce IFNs. Examples of LMP-1 expressing cell lines suitable or use in the present invention include BL-41-EBV, Sav III, IB4 others known in the art (5, 11, 12, 33, 48). In one embodiment, the portion of a herpesvirus genome is transfected into a cell isolated from a subject, such as a PBMC. Furthermore, LMP-1 from a herpesvirus may be introduced into any cell by the techniques described below. The cells may express full length (wild type) LMP-1 or any portion thereof sufficient to prime the cell for IFN production. In one embodiment, the cell expresses LMP-1 that comprises a least one CTAR domain (79).

The cells used in the methods of the present invention can include any mammalian cell, but are preferably human cells, especially when the protein produced is for use in humans. Particularly preferred cells include any freshly isolated cells or cell lines derived from human leukocytes, including peripheral blood mononuclear cells. In a more preferred embodiment, the cells are human lymphocytes, with B lymphocytes being particularly preferred. Methods of isolating cells, including B lymphocytes, from blood are well known in the art. Such methods can include any method of obtaining a sample of blood or plasma from an individual, followed by techniques for isolating or purifying the desired cells from the blood or plasma sample. Cells can also be provided in the form of pre-established cultures or cell lines, if desired.

The robust induction of IFN may result from the infection or superinfection of primed cells by many viruses. Any virus that induces the production of IFN from a cell upon infection may be used. Examples of viruses suitable for use in the present invention include, but are not limited to, Sendai virus, Newcastle disease virus (NDV) and vesicular stomatitis virus (VSV). In addition, a virus-like stimulus (e.g., a stimulus that mimics viral infection in that it induces a cellular response similar to that induced by viral infection) can be used to induce IFN production. Such virus-like stimuli are known in the art. For example, exposure of PBMCs to dsRNA may induce the production of IFNs. In one embodiment, IFN production may be induced by exposing a cell to double-stranded RNA.

Some embodiments of the present invention relate to a method for the production of IFN. The interferon produced by the methods of the present invention can include a type I interferon, including, but not limited to, IFN-α, IFN-β, IFN-θ, or INF-ω, including subtypes or species thereof (e.g., IFN-α2a), with production of IFN-α or IFN-β, including subtypes or species thereof, being particularly preferred. Combinations of any of the IFNs listed above are also within the scope the invention. The present invention also includes IFNs produced by the methods of the present invention.

The present inventors have discovered that herpesvirus-immortalized human cells produced significantly higher amounts of IFN in vitro than corresponding, non-immortalized blood leukocytes or cultured cells on a per cell basis, making the system invaluable for provision of enhanced quantities of IFN for therapeutic use. The immortalized cells can be propagated in vitro for many generations, and IFN synthesis can be induced to high levels using conventional induction methods. Moreover, immortalized cells (such as EBV-immortalized PMBCs) can be easily established using cells isolated from any individual's blood, and so the system of the present invention can be readily used to produce personalized IFN that is particularly suitable for use in the individual from which the cells were obtained. IFN produced commercially prior to the present invention, including natural interferon, was not specific to an individual. Such IFNs are naturally glycosylated by the human host cell and thus reduce issues associated with natural antibodies, and this advantage is expected to be enhanced when the IFNs are produced from a patient's own cells. The IFNs produced by the system are easy to purify from the immortalized cells, and so the administration of the IFN produced by the system for therapeutic purposes is safe.

The present invention thus provides methods to produce personalized IFN comprising obtaining a cell from a subject, priming the cell for interferon production by infection of the cell with a herpesvirus or transfection of the cell with a portion of a herpesvirus genome, infecting the cell with a virus to induce the cell to produce IFN, and recovering the IFN from the cell. In some embodiments, the personalized IFN may be used to treat the patient who provided the cells for the IFN production.

While not wishing to be bound by any particularly theory, personalized IFNs may provide numerous benefits over existing, non-personalized IFNs currently in use. These benefits include, but are not limited to, higher efficiency, lower toxicity and longer circulation time when the personalized IFNs are administered to the specific individual whose cells were used to make the IFN. Currently, the majority of IFNs on the market are recombinant IFNs composed of one or few subtypes of IFNs. However, deletion of one subtype (e.g., IFN-β) from the mouse genome leads to decreased capability to fight viral diseases (17). Thus, all IFN subtypes are not the same and the maximum therapeutic benefit may require the combination of all IFN subtypes, as produced by the personalized system described herein. Further, some recombinant IFN-resistant patients (i.e., those resistant to one subtype of IFN) with hairy cell leukemia have been treated effectively with natural IFNs (multiple IFNs derived from human cells), suggesting that “natural” IFNs such as those produced by the personalized system are better than recombinant ones.

Some IFN subtypes are modified by glycosylation, and different persons may have different modifications. The personalized IFNs are natural IFNs with multiple subtypes, and posses identical glycosylation patterns as the specific individual cell donor. Taken together, these benefits indicate that personalized IFNs will exhibit higher efficiency when administered to the specific individual.

The personalized IFNs are also expected to be less toxic than non-personalized IFNs. Because the personalized IFNs are from the specific individual's own cells, the side effects (e.g., antibody and inflammatory responses resulting from the administration of a non-personalized natural IFN or a recombinantly produced IFN should be minimized. The patient's failure to recognize the personalized IFN as a foreign or non-self protein should also result in a longer half-life for the personalized IFN. Thus, the circulation time of the administered IFNs in the subject is expected to be longer than conventional versions of IFNs.

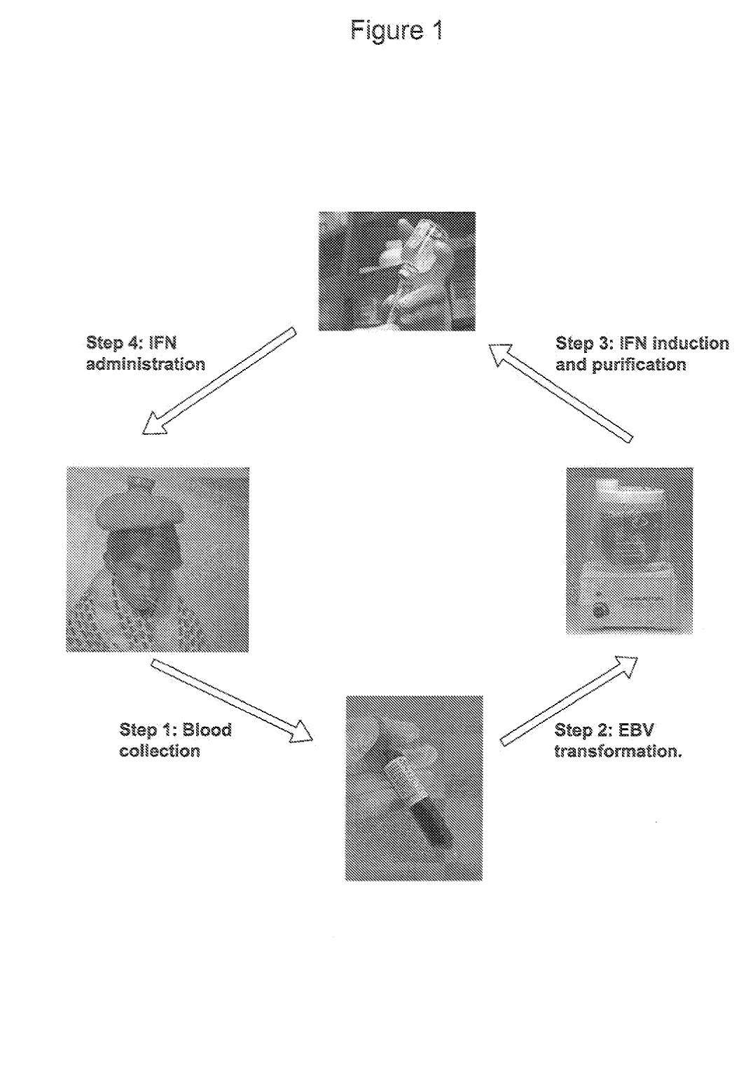

Accordingly, one aspect of the present invention provides a system for the production of personalized IFN. A generalized depiction of one embodiment of the invention is illustrated in FIG. 1, in which four steps are used to produce the personalized IFN. In step 1, blood (e.g., 5 to 10 mls) is collected from a subject and PBMCs are isolated from the blood. In step 2, the isolated PBMCs are infected with a herpesvirus (in this example, EBV), and the resulting immortalized lymphoblastoid cell lines (LCLs) are grown in a tissue culture system. In step 3, the LCLs, now primed for the production of IFNs by the EBV transformation, are induced to produce IFNs by superinfection with Sendai virus. The resulting IFNs may be purified by standard protocols. Finally, in step 4, the purified IFNs are administered to the same subject who donated the PBMCs. By this system, IFNs generated by a specific individual's own cells can be used to treat the same individual.

The systems and methods for the production of personalized IFNs described herein may use any of the components or parameters discussed above. For example, any herpesvirus, portion of the genome thereof, or virus for inducing IFN production discussed above is suitable for the production of personalized IFNs.

Because herpesvirus transformation immortalizes the individual's cells, they may be grown in culture for any length of time to allow for the continuous production of personalized IFNs. The cells may be cultured according to the conditions described below in any size culture vessel. For example, one-liter and five-liter culture vessels can be used to produce large amounts of both personalized IFNs from an individuals's cells or non-personalized IFNs from a primed cell line. In addition, the cells producing the personalized or non-personalized IFNs may be frozen and thereby stored until needed. Techniques for freezing, thawing and culturing cells and cell lines are well known in the art.

By way of example, in one embodiment of the present invention, Epstein Barr Virus (EBV) type III latency cells (i.e., cells that are latently infected with EBV showing the type III pattern of gene expression) are primed to produce robust levels of endogenous IFNs upon viral superinfection. The inventors have shown that the viral protein LMP-1 is sufficient for the priming action in EBV-infected cells. LMP-1-mediated activation of NFκB is also involved with this priming effect, and may operate in part through activation of IFN regulatory factor 7 (IRF-7). In summary, the inventors have shown that LMP-1 primes latently EBV-infected cells for IFN production, and that the antiviral property of LMP-1 may be an intrinsic part of the EBV latency program, assisting in the establishment and/or maintenance of viral latency.

LMP-1 is the principal oncoprotein required for EBV transformation of human B cells and establishment of latency in vitro. LMP-1 is an integral membrane protein with six transmembrane-spanning domains with a long C-terminal domain, which is located in the cytoplasm (34, 39). Two C-terminal activator regions (CTARs) have been identified to initiate signal transduction (79). LMP-1 acts as a constitutively active receptor-like molecule that does not need the binding of a ligand (23). LMP-1 appears to be a central effector of altered cell growth, survival, adhesive, invasive and antiviral potential (19, 42, 65, 66, 69). Previously, it has been shown that LMP-1 induces several antiviral IFN stimulated genes (ISGs) without trigging IFN production and LMP-1 possesses antiviral activity (70).

The amino acid sequence of LMP-1 is provided as SEQ ID NO:1. The CTAR-1 domain is represented by amino acid residues 194-232 of SEQ ID NO:1, while the CTAR-2 domain is represented by amino acid residues 351-386 of SEQ ID NO:1. Additional herpesvirus proteins expressed in type III latency (i.e., EBNA-1, EBNA-2, EBNA-3A, EBNA-3B, EBNA-3C, EBNA-LP, LMP-2A and LMP-2B) and the amino acid and nucleic acid sequences of each are well known in the art. The sequence of entire genome of EBV and the sequences of proteins encoded therein can be found at Genbank Accession No. AJ507799. The information provided in Genbank Accession No. AJ507799 is incorporated herein by reference in its entirety. In addition, detailed information regarding the structure of the EBV genome and proteins encoded thereby is found in: Arrand et al., Nucleic Acids Res. 9 (13), 2999-3014 (1981); Kozak, Nucleic Acids Res. 9(20), 5233-5252 (1981); Deininger et al., J. Cell. Biochem. 19 (3), 267-274 (1982); Farrell et al., EMBO J. 2(8), 1331-1338 (1983); Farrell et al., Proc. Natl. Acad. Sci. U.S.A. 80 (6), 1565-1569 (1983); Bankier et al., Mol. Biol. Med. 1 (1), 21-45 (1983); Sequin et al., Mol. Biol. Med. 1 (3), 369-392 (1983); Jeang and Hayward, J. Virol. 48 (1), 135-148 (1983); Bankier et al., Mol. Biol. Med. 1 (4), 425-445 (1983); Jones et al., EMBO J. 3 (4), 813-821 (1984); Biggin et al., EMBO J. 3 (5), 1083-1090 (1984); Yates et al., Proc. Natl. Acad. Sci. U.S.A. 81 (12), 3806-3810 (1984); Gibson et al., Nucleic Acids Res. 12 (12), 5087-5099 (1984); Baer et al., Nature 310, (5974), 207-211 (1984); Bodescot et al., Nucleic Acids Res. 15 (14), 5887 (1987); Laux et al., EMBO J. 7 (3), 769-774 (1988); Parker et al., Virology 179 (1), 339-346 (1990); and de Jesus et al., J. Gen. Virol. 84 (PT 6), 1443-1450 (2003), each of which is incorporated herein by reference in its entirety.

It is understood that different types and strains of viruses exist that may possess allelic variants of the proteins described herein. For example, at least two types of EBV, EBV1 and EBV2, are known, each possessing distinct alleles for latency genes. The present invention includes allelic variants and other homologues of the proteins and nucleic acids disclosed or referenced herein. In one embodiment of the invention, homologues of a given protein (which can include related proteins from other organisms or modified forms of the given protein) are encompassed for use in the invention. Homologues of a protein encompassed by the present invention can comprise an amino acid sequence that is at least about 35% identical, and more preferably at least about 40% identical, and more preferably at least about 45% identical, and more preferably, at least about 50% identical, and more preferably at least about 55% identical, and more preferably at least about 60% identical, and more preferably at least about 65% identical, and more preferably at least about 70% identical, and more preferably at least about 75% identical, and more preferably at least about 80% identical, and more preferably at least about 85% identical and more preferably at least about 90% identical, and more preferably at least about 95% identical, and more preferably at least about 96% identical, and more preferably at least about 97% identical, and more preferably at least about 98% identical, and more preferably at least about 99% identical, or any percent identity between 35% and 99%, in whole integers (i.e., 36%, 37%, etc.) to an amino acid sequence disclosed or referenced herein. Preferably, the amino acid sequence of the homologue has a biological activity of the wild-type or reference protein. The invention also encompasses nucleic acid molecules encoding the protein homologues described above.

In one embodiment, a protein of the present invention comprises an amino acid sequence that is less than 100% identical to SEQ ID NO:1 (i.e., a homologue). In another aspect of the invention, a homologue according to the present invention has an amino acid sequence that is about 99% identical to SEQ ID NO:1, and in another embodiment, is about 98% identical to SEQ ID NO:1, and in another embodiment, is about 97% identical to SEQ ID NO:1, and in another embodiment, is about 96% identical to SEQ ID NO:1, and in another embodiment, is about 95% identical to SEQ ID NO:1, and in another embodiment, is about 94% identical to SEQ ID NO:1, and in another embodiment, is about 93% identical to SEQ ID NO:1, and in another embodiment, is about 92% identical to SEQ ID NO:1, and in another embodiment, is about 91% identical to SEQ ID NO1, and in another embodiment, is about 90% identical to SEQ ID NO:1, and so on, in increments of whole integers. In certain embodiments, the homologue protein has a biological activity of the wild-type or reference protein.

In accordance with the present invention, an isolated nucleic acid molecule is a nucleic acid molecule (polynucleotide) that has been removed from its natural milieu (i.e., that has been subject to human manipulation) and can include DNA, RNA, or derivatives of either DNA or RNA, including cDNA. As such, “isolated” does not reflect the extent to which the nucleic acid molecule has been purified. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding, a protein. An isolated nucleic acid molecule of the present invention can be isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a protein of the present invention or to form stable hybrids under stringent conditions with natural gene isolates. An isolated nucleic acid molecule can include degeneracies. As used herein, nucleotide degeneracies refers to the phenomenon that one amino acid can be encoded by different nucleotide codons. Thus, the nucleic acid sequence of a nucleic acid molecule that encodes a protein of the present invention can vary due to degeneracies. It is noted that a nucleic acid molecule of the present invention is not required to encode a protein having protein activity. A nucleic acid molecule can encode a truncated, mutated or inactive protein, for example. Such nucleic acid molecules and the proteins encoded by such nucleic acid molecules are useful in as probes and primers for the identification of other proteins. If the nucleic acid molecule is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

One embodiment of the present invention relates to a recombinant nucleic acid molecule which comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the protein or homologue thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

The amount of IFNs produced by viruses or double stranded RNA (dsRNA) can be increased robustly by treating cells with IFN before infection, a phenomenon known as IFN priming (21, 28, 61). The stimulation of IFN production observed upon priming results from an increase in the rate of IFN gene transcription and may involve an IFN-inducible factor(s) (18, 47).

The mechanism of the transcriptional activation of IFNs has been under intensive investigation. One of the major players in the IFN production is IFN regulatory factor 7 (IRF-7). IRF-7 was cloned in the context of EBV latency and has an intimate relation with EBV (74-80). IRF-7 is inducible by type I IFNs and can be further activated by phosphorylation and nuclear translocation upon viral infection, and activated IRF-7 is partially responsible for the robust transcriptional activation of IFNs (4, 40, 41, 68, 78).

While it has been established that IFN primes cells for robust IFN production upon viral infection, the present inventors have demonstrated that EBV type III latency cells are also primed for robust IFN production. Because current evidence suggests that EBV latency cells cannot produce type I IFNs, the priming effect of type III latency cells was hypothesized to be due to viral protein(s) expressed during latency. The present inventors have now identified EBV LMP-1 as a gene capable of priming cells for robust IFN production (e.g., in EBV-infected B lymphocytes).

The stimulation of IFN production observed upon priming results from an increase in the rate IFN gene transcription and involves an IFN-inducible factor(s) (18, 47). It seems likely that IRF-7 is an important factor in the priming action of IFN because IRF-7 is inducible by IFN and IRF-7 is responsible for the robust and wide variety of type I IFNs production (38, 78). It is of note that there is a striking similarity between IFN-primed and LMP-1-expressing cells; the high expression levels of ISGs including IRF-7. Although LMP-1 is able to activate IRF-7, NF-κB, and ATF-2, three critical factors for type I IFNs production, LMP-1 has not been shown to activate type I IFNs production in viral latency, but selectively induces ISGs (70, 74, 79).

NF-κB is an essential factor in type I IFN production (56, 57). The present inventors have demonstrated that activation of NF-κB by LMP-1 is involved in LMP-1-mediated priming action. LMP-DM failed to activate NF-κB and failed to prime cells. Blocking LMP-1-mediated NF-κB activation by IκB expression eliminated the priming function. NF-κB alone was able to induce the priming state. Thus, the present inventors have shown that NF-κB is necessary and sufficient for the LMP-1-mediated priming effect in human B lymphocytes.

While not wishing to be bound by any one theory, the induction of ISGs, or IRF-7 in particular, may also be one of the molecular bases for the priming property of EBV latency cells. First, there is a great deal of correlative data to suggest that high IRF-7 is associated with primed latency cells. IRF-7 is highly expressed in EBV type III latency cells, such as in BL41-EBV and Sav III (74, 77). The high levels of IRF-7 in viral latency is primarily due to the expression of LMP-1 (74, 79). The present inventors have demonstrated that LMP-1 is able to induce IRF-7 and is able to prime cells for IFN production; however LMP-DM, which failed to induce the expression of IRF-7 (79), was unable to significantly induce IFN-β. Second, the present inventors have shown that NF-κB is necessary and sufficient to induce priming action in EBV latency cells. However, NF-κB alone is able to induce IRF-7 in DG75 cells, and LMP-1-mediated induction of IRF-7 also requires NF-κB (79). Thus, it was hypothesized that IRF-7 is also involved in NF-κB-mediated priming processes. Third, the present inventors have demonstrated that ectopic expression of IRF-7 alone is sufficient to achieve higher levels of IFN production. Based on the well-established role of IRF-7 in IFN production and the fact LMP-1 induces IRF-7, IRF-7 may be one of the factors in LMP-1-mediated priming process of EBV latency cells. However, as demonstrated herein, LMP-1-mediated priming appears more efficient than that by overexpression of IRF-7, suggesting that an additional factor(s) may also involved. The observation that BL41-EBV expressed higher levels of several cellular factors that are well-known players in IFN production than BL41 cells based on microarray experiments supports this concept.

One embodiment of the present invention relates to a cell-system and method for the production of IFN. The system comprises cells that have been infected with a herpesvirus such as EBV (or transfected with suitable portion of the genome thereof) that primes cells for IFN production. In one embodiment, instead of infecting the cells with the entire herpesvirus genome, the cell is transfected with a portion of the herpesvirus genome or an herpesvirus gene or genes sufficient to prime IFN production by the cell (e.g., LMP-1). In certain embodiments, the herpesvirus genome or portion thereof is from EBV.

Cells used in the present invention are cultered under appropriate culture conditions for the cell type. Such conditions include an effective medium in which the cell can be cultured, including a medium suitable for culture of animal cells and particularly human cells. Such a medium comprises a base medium plus assimilable sources of carbon, nitrogen and micronutrients (e.g., a serum source, growth factors, amino acids, antibiotics, vitamins, reducing agents, and/or sugar sources). It is noted that completed mediums necessary for animal cell growth are commercially available, and some media are available for particular types of cell culture. Suitable culture conditions are described in the Examples. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art.

Cells can be infected with a herpesvirus such as EBV, or a selection of genes therefrom that are sufficient for the priming of IFN production by the cell (e.g., LMP-1), using any suitable protocol for transfection of human cells. Such methods include, but are not limited to, viral transfection, direct electroporation, transduction, infection, or other suitable techniques for the introduction of viruses and nucleic acid molecules into a cell. According to the present invention, the term “transfection” is used to refer to any method by which an exogenous nucleic acid molecule (i.e., a recombinant nucleic acid molecule) can be inserted into a cell. The term “transduction” is a specific type of transfection in which genetic material is transferred from one source to another, such as by a virus (e.g., a retrovirus) or a transducing bacteriophage. The term “transformation” can be used interchangeably with the term “transfection” when such term is used to refer to the introduction of nucleic acid molecules into microbial cells, such as bacteria and yeast. In microbial systems, the term “transformation” is used to describe an inherited change due to the acquisition of exogenous nucleic acids by the microorganism and is essentially synonymous with the term “transfection.” However, in animal cells, transformation has acquired a second meaning that can refer to changes in the growth properties of cells in culture after they become cancerous or immortalized, for example. Therefore, to avoid confusion, the term “transfection” is preferably used herein with regard to the introduction of exogenous nucleic acids into animal cells. Therefore, the term “transfection” will be used herein to generally encompass transfection or transduction of animal cells, and transformation or transduction of microbial cells, to the extent that the terms pertain to the introduction of exogenous nucleic acids into a cell. Transfection techniques include, but are not limited to, transformation, transduction, particle bombardment, diffusion, active transport, bath sonication, electroporation, microinjection, lipofection, adsorption, infection and protoplast fusion.

IFNs produced by the system and method of the present invention can be recovered from the cells by any suitable method. For example, cell supernatants or lysates can be filtered and/or entrifuged to remove cells, cell debris and other particulate matter, and the product can be recovered from the remaining supernatant by conventional methods, such as, for example, ion exchange, chromatography, extraction, solvent extraction, phase separation, membrane separation, electrodialysis, reverse osmosis, distillation, chemical derivatization and crystallziation. Antibodies against IFN proteins and species thereof are available in the art and can be used in a variety of immune-based methods of protein separation and purification. If desired, different IFN species can be separately isolated from the production cell.

Once the IFNs are isolated and/or purified from the production cells and culture, the interferons can be used in a research method, or to raise antibodies, or in a preferred method, are administered to an individual who would potentially benefit from the therapeutic administration of an IFN. Such an individual includes an indivudal who has a disease or condition including, but not limited to, hepatitis C infection, hepatitis B infection, cancer (e.g., kidney, malignant melanoma, multiple myeloma, carcinoid tumors, Karposi's sarcoma, and some types of lymphoma and leukemia), multiple sclerosis (MS), and other diseases of viral, malignant, angiogenic, allergic, inflammatory, and fibrotic origin that may benefit from IFN administration.

According to the present invention, IFN is typically administered to a patient in a composition. In addition to the IFN, the composition can include, for example, a pharmaceutically acceptable carrier, which includes pharmaceutically acceptable excipients and/or delivery vehicles, for delivering the IFN to a patient. As used herein, a pharmaceutically acceptable carrier refers to any substance suitable for delivering a therapeutic composition useful in the method of the present invention to a suitable in vivo or ex vivo site. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

According to the present invention, an effective administration protocol (i.e., administering a composition of the present invention in an effective manner) comprises suitable dose parameters and modes of administration that result in delivery of IFN to the patient, preferably so that the patient obtains some measurable, observable or perceived benefit from such administration. Suitable methods of administering an IFN or a composition comprising IFN to a subject include any route of in vivo administration that is suitable for delivering the composition. The preferred routes of administration will be apparent to those of skill in the art, depending on the type of delivery vehicle used and the disease or condition experienced by the patient. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue.

A preferred single dose of IFN typically comprises between about 0.01 and about 10 mg, including any dosage in between, in increments of 0.01 mg (e.g., 0.01 mg, 0.02 mg, 0.03 mg, . . . ). IFN is typically administered to a patient every day, every other day, or as needed, but conventional dosages and administration protocols are well-known in the art for various conditions that are currently treated using IFN (recombinant or natural).

A therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). A beneficial effect can easily be assessed by one of ordinary skill in the art and/or by a trained clinician who is treating the patient. The term, “disease” refers to any deviation from the normal health of a subject and includes a state when disease symptoms are present, as well as conditions in which a deviation (e.g., infection, gene mutation, genetic defect, etc.) has occurred, but symptoms are not yet manifested.

Exemplary methods according to the invention are present below. The following examples are provided for the purpose of illustration and are not intended to limit the scope of the present invention.

### EXAMPLES

The following Materials and Methods were used in the examples below.

Plasmids, Antibodies and Viruses

Expression plasmids of LMP-1 and its signaling defective mutant, LMP-DM, IRF-7 and its DNA binding mutant IRF7-K92E, have been described previously (71, 79). Expression plasmid pAG155 was used for expression of EBNA-2 (75). NF-κB related plasmids: p65, p50, IκB expression plasmids, plus the NF-κB reporter construct have been previously described (26, 64). EBNA-2 antibody (PE2) and LMP-1 Ab (CS1-4) were purchased from Dako. STAT-1 antibody was purchased from Santa Cruz Biotechnology. Tubulin antibody was purchased from Sigma. Anti-Sendai virus antibody was purchased from US Biologicals, Inc. Recombinant human IFN-α was purchased from Schering-Plough. Sendai virus stock was purchased from Spafas, Inc. For virus infection, 200 HA units/ml of Sendai virus were added to the target cells for 6 hours, and cells were then collected for RNA isolation. However, seven-hour infection was used for examining the endogenous IFN production in the media.

Cell Culture, Transient Transfection, and Isolation of Transfected Cells

DG75 is an EBV-negative Burkitt's lymphoma cell line (5). BL41 is an EBV-negative BL line. BL41-EBV was generated by in vitro infection of BL41 with EBV B95-8 strain (11). Sav I and Sav III are genetically identical cell lines that differ only in their latency types (48). Akata (type I) and IB4 (Type III) are EBV-positive cell lines. These cells were maintained in RPMI-1640 plus 10% fetal bovine serum (FBS). 293 cells are human fibroblasts and are maintained in DMEM plus 10% FBS. Electroporation (320V; 925 μF) was used for transfection of the B cells as described previously (74, 75, 79). A total of 10 μg of DNA were used for transfection of DG75 cells. 1 μg of LMP-1-expression plasmids were always used in transfection to achieve similar LMP-1 expression levels in transfected and EBV type III latency cells. Enrichment for CD4 positive cells was performed with the use of anti-CD4-antibody conjugated to magnetic beads according to the manufacturer's recommendation (Dynal, Inc.). DG75 cells were transfected with CD4 expression plasmids and other plasmids. One day after the transfection, CD4-positive cells were isolated with the use of Dynabeads CD4 (Dynal Inc.) The transfected cells were incubated with Dynabeads-CD4 at 72 μl beads/10⁷ cells for 20-30 minutes at 4° C. with gentle rotation. CD4-positive cells were isolated by placing the test tubes in a magnetic separation device (Dynal magnet). The supernatant was discarded while the CD4-positive cells were attached to the wall of the test tube. The CD4-positive cells were washed 4-5 times in PBS plus 2% FBS, and resuspended in 100 μl RPMI 1640 plus 1% FBS. Cells were detached from the Dynabeads CD4 by incubating for 45-60 minutes at room temperature with 10 μl of DETACHaBEAD (Dynal). The detached beads were removed by using the magnet separation device. The released cells were washed 2-3 times with 500 μl RPMI 1640 plus 10% FBS, and resuspended in RPMI 1640 plus 10% FBS at 5×10⁵ cells/ml. The isolated cells were used to extract total RNA or prepare cell lysates immediately, or recovered overnight before infection by viruses.

Western Blot Analysis with Enhanced Chemiluminescence (ECL)

Separation of proteins on SDS-PAGE was carried out following standard protocols. After the proteins were transferred to a nitrocellulose of Immobilon membrane, the membrane was blocked with 5% non-fat dry milk in TBST (50 mM Tris-HCl pH 7.5, 200 mM NaCl, 0.05% Tween-20) at room temperature for 10 minutes. It was then washed briefly with TBST, and incubated with the primary antibody in 5% milk in TBST for 1 h at room temperature, or overnight at 4° C. After washing with TBST three times (10 minutes each), the membrane was incubated with the secondary antibody at room temperature for 1 h. It was then washed three times with TBST, treated with ECL detection reagents (Amersham, Piscataway, N.J.), and exposed to Kodak XAR-5 film.

RNA Extraction, RNase Protection Assays (RPA), and Reporter Assays

Total RNA was isolated from cells using the RNeasy Total RNA Isolation Kit (Qiagen, Valencia, Calif.) or Trizol extraction. RPA was performed with 10 μg of total RNA using the RNase Protection Assay Kit II (Ambion, Houston, Tex.) at 55° C. Sometimes, gradient temperatures were performed for RPA when difficulties in RPA were encountered (72). The GAPDH probe was from US Biochemicals, Inc. The probe for IFN-β have been previously described (51). The luciferase reporter assays were performed using the assay kit from Promega according to manufacturer's recommendation.

Removal of Sendai Virus from Culture Medium

The removal of Sendai virus was achieved by the use of 15-nm Planova Virus Removal Fitlers (Asahi Kasei Pharma Corporation). Seven hours after Sendai virus infection, cell culture media were collected and passed through the Planova filters following the manufacturer's protocol. To test if the filtered media contain Sendai viruses, the filtered conditional media is used to treat fresh BL41 cells, and cell lysates made 24 hours later. Western blot with anti-Sendai antibody was used to detect any Sendai proteins in cell lysates. Sendai virus treated (20 HA units/ml) cell lysates were used as positive controls.

IFN-α Measurement and Functional Assay

The concentration of IFN-α was determined by a commercially available Human Interferon Alpha (Hu-IFN-α), ELISA Kit (PBL Biomedical Laboratories; Catalog #41100) according to the manufacturer's recommendations. The kit is able to detect human IFN-αA, α2, αA/D, αD, αK, and α4b. Samples were examined in triplicates. To test the functions of IFNs in cell culture media, the conditional media were first passed through a 15-nm filter to remove Sendai viruses. These media were then used to treat fresh BL41 cells at desired dilutions for 24 hours and cell lysates were used for the detection of STAT-1 expression. The human IFN-α neutralization antibody (RDI-PB31130) was purchased from Fitzgerald Industries Intl. The antibody is able to react with several kinds of IFN-α subtypes, but not IFN-β and -ω. 1000,000 neutralization units/ml were added to BL41 cells along with suitable concentration of conditional media, and incubated for 24 hours. The expression of STAT-1 in BL41 cells is then measured by Western blot analyses.

### Example 1

The following example demonstrates the generation of personalized IFNs using a method of the present invention.

FIG. 1 illustrates a system for the generation of personalized IFNs. This system harnesses at least two properties of the herpesvirus EBV: 1) the ability to immortalize primary B lymphocytes; and 2) the ability to prime its latency cells for IFN production.

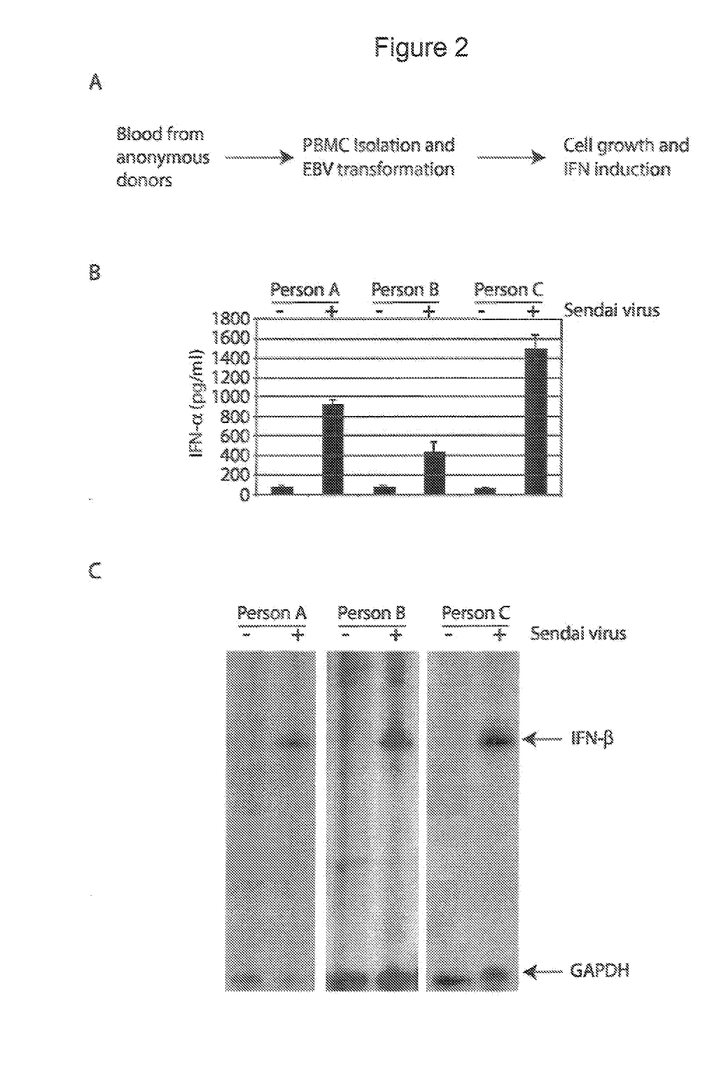

To test the system, peripheral blood mononuclear cells (PBMCs) from three donors (Persons A, B and C, respectively) were used to generate personalized lymphoblastoid cell lines (LCLs), as illustrated by FIG. 2A. PBMCs were isolated from each donor's blood using Ficoll-Paque Plus (Amersham) according to standard protocols. The cells were exposed to 0.45-μm-filtered virus supernatants from EBV stocks, and the newly infected lymphocytes were seeded into growth medium at 2×10⁵ cells/ml in 96-well plates. Five weeks later, LCL outgrowth was evident in most wells containing EBV-infected cells. The proliferating cells were collected and expanded into T25 flasks. When 10 ml or more cells were obtained, Sendai viruses (200 HA units per ml) were used to infect these cells and the production of IFNs was determined.

Seven hours after the Sendai virus infection, ELISA was used for the detection of IFN-α proteins in the culture media as described above. As shown in FIG. 2B, the Sendai virus-infected LCLs produced large amounts of IFN-α. The uninfected cells produced much lower levels of IFN-α, in agreement with previous reports that EBV transformed cells produce low levels of IFNs (2, 6).

In addition, induction of IFN-β mRNA in LCLs infected with Sendai virus for six hours was examined by RNase protection assays as described above. The use of IFN-β as a general indicator for type I IFN production is well established in the field. As shown in FIG. 2C, IFN-β mRNA was highly induced upon infection of Sendai virus. Taken together, these data demonstrate that the system can produce personalized type I IFNs.

### Example 2

The following example demonstrates that EBV type III latency cells have a high capacity to produce IFN-β when superinfected.

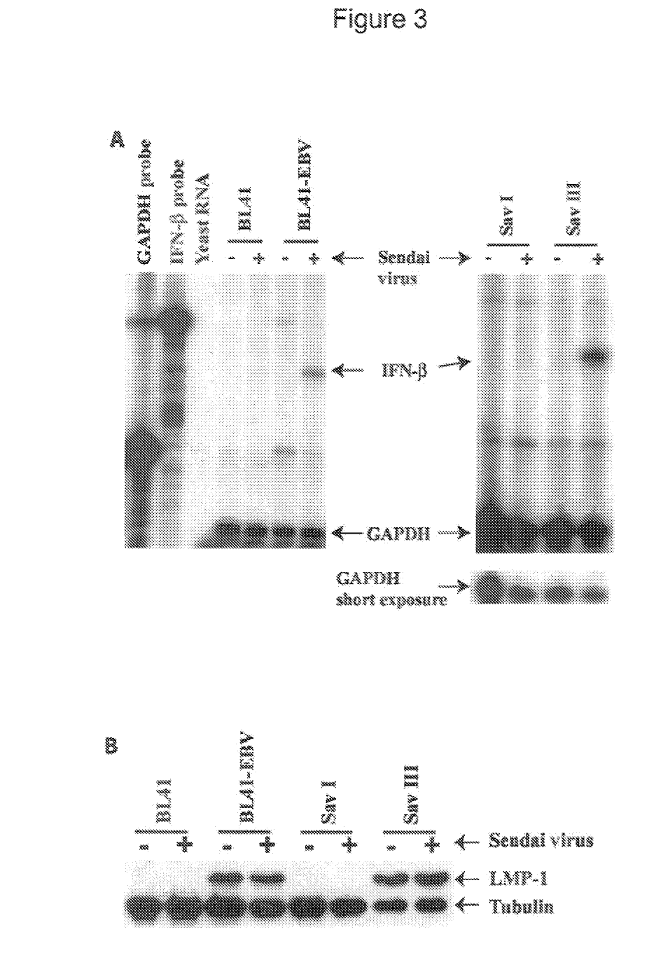

EBV type III latency cells express high levels of IRF-7 and other IFN stimulated genes (ISGs), similar to IFN treated cells. Because IFN can prime the cell for robust IFN production, whether these EBV latency cells are primed for higher IFN production upon viral superinfection was examined. BL41 is an EBV-negative Burkitt's lymphoma line, and BL41-EBV is its EBV-infected derivative with type III latency. Sav I (type I latency) and Sav III (type III latency) are genetically identical sister cell lines that differ only in their types of latency. These two pairs of cell lines were used to address the presence of EBV type III latency on the production of IFN. Sendai virus (200 HA units/ml) was used to infect these cells and total RNA was isolated 6 hours post infection. RPA experiments were used for the detection of IFN-β RNA production as described above, with yeast RNA used as a negative control. As shown in FIG. 3A, while BL41 and Sav I cells had little or no detectable IFN-β production, high levels of IFN-β RNA were observed in BL41-EBV and Sav III cells, both of which are type III latency cells with LMP-1 expression (FIG. 3B). IFN-β production in Akata (type I latency) and IB4 (type III latency) cells upon on Sendai infection was also tested. While IB4 could produce high levels of IFN-β, Akata cells failed to produce significant amount (data not shown). These data demonstrate that the induction of IFN-β upon viral infection is enhanced in EBV type III latency cells.

### Example 3

The following example demonstrates that EBV type III latency cells produce functional type I IFNs upon superinfection.

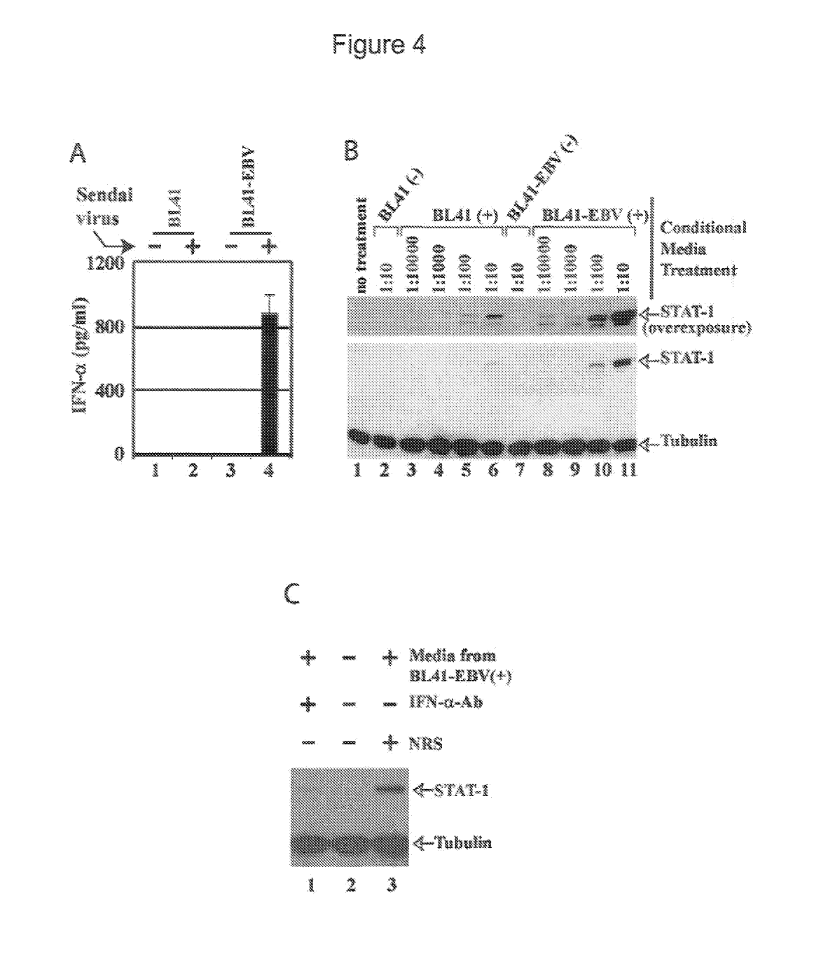

Type I IFNs are a family of IFN-α, one IFN-β, and one IFN-ω. However, using IFN-β as an indicator for type I IFN production is well established and appreciated in the field (4, 40, 68, 71). Thus, the results above suggest that type I IFNs were robustly induced in EBV latency cells by superinfection. Because IFN-β RNA is synthesized in EBV-infected cells, and because EBV and other herpesviruses encode genes to shut off host gene expression (8, 13, 15, 29, 59), we examined if IFN proteins are synthesized and secreted properly in BL41-EBV cells superinfected with Sendai virus as above. ELISA was used for the detection of IFN-α proteins in the culture media. As shown in FIG. 4A, the Sendai virus-infected BL41-EBV cells produced a large amount of IFN-α. However, uninfected BL41-EBV cells and Sendai infected BL41 cells produced undetectable levels of IFN-α, in agreement with the data in FIG. 3. These results indicate that IFNs are properly synthesized and secreted from EBV latency cells.

Whether these secreted IFNs were functional was then examined. BL41 and BL41-EBV were infected with Sendai (200 HA units per ml) for 7 hours along with uninfected controls. The media from Sendai virus infected or uninfected BL41-EBV cells were collected and passed through 15-nm filters to eliminate Sendai viruses (62). We confirmed that the filtered media did not contain detectable Sendai viruses as determined by viral protein expression (data not shown). These conditional media were then used to treat fresh BL41 cells at various dilutions, and the expression of STAT-1 was determined 24 hours later by preparing cell lysates and performing western blot analyses with STAT-1 and tubulin antibodies. STAT-1 was used as an indicator of IFN functions because STAT-1 is highly inducible by IFN and Sendai virus blocks IFN signaling (24, 25, 35, 36). Therefore, a successful induction of STAT-1 indicates the presence of functional IFNs and absence of Sendai viruses. As shown in FIG. 4B, the conditional medium from BL41-EBV-superinfected cells was approximately 10 fold more potent than that from BL41-infected cells for induction of STAT-1. The apparent discrepancy between FIG. 4A (column 2) and FIG. 4B (lanes 3-6) might be due to the fact that the ELISA kit only detects a subset of IFN-α (see above for details), and/or viral superinfection has produced other secretable factors that are capable of inducing STAT-1. For example, LMP-1 has been shown to produce a soluble factor(s) that induces STAT-1 (53).

To confirm that IFNs in the conditional media were responsible for the induction of STAT-1, the experiments were repeated in the presence of a IFN-α neutralization antibody. As shown in FIG. 4C, the human IFN-α neutralization antibody partially inhibited the induction of STAT-1. Therefore, type III latency cells had produced functional IFNs and IFN-β RNA was a good indicator for the robust type I IFN production in this system. Data in FIGS. 3 and 4 collectively demonstrate that EBV type III latency cells are primed for type I IFN production.

### Example 4

The following example demonstrates that LMP-1 primes cells for the expression of IFNs.

Previous results suggest that unstimulated EBV latency cells do not produce IFNs. (14, 31, 70, 73). For example, the results depicted in FIG. 4 show that EBV latency cells (BL41-EBV) did not produce significant amounts of IFNs without viral infection (FIG. 4A, column 3 and FIG. 4B, lane 7). Thus, the priming effect of EBV latency cells may be the function of an EBV latency protein(s) rather than the production of IFN by latency cells per se.

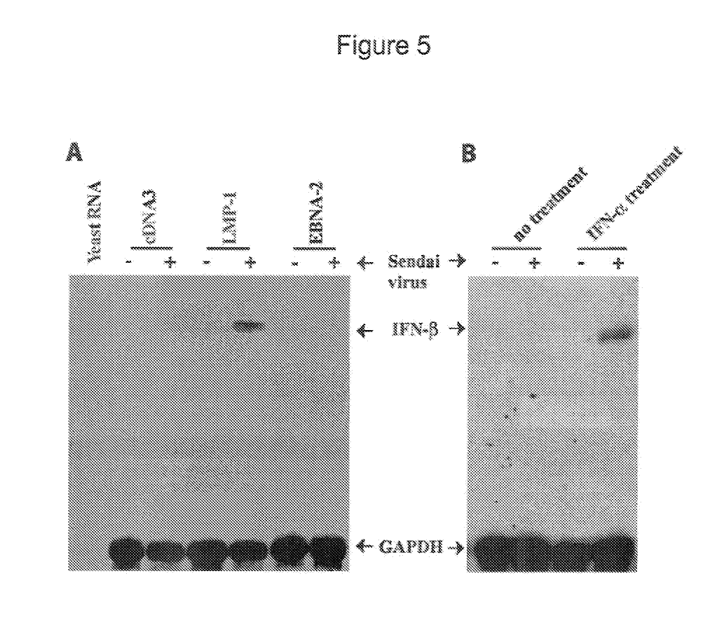

There are several viral genes expressed in EBV type III latency. However, because LMP-1 induces IRF-7 and other ISGs, and has antiviral effect without inducing IFNs (70, 73, 74, 79), and because IRF-7 is a master gene for IFN production (32), whether LMP-1 can prime cells for the expression of IFNs was examined. In addition, EBNA-2, the primary inducer of LMP-1 (1, 22, 63, 67), might play a role for the priming of the latency cells. DG75 cells, which are EBV-negative Burkitt's lymphoma cells, were used for the experiments because of transfection efficiency. Vector, LMP-1 or EBNA-2 and a CD4-expression plasmid were transfected into cells. Transfected cells were enriched and split into two wells: one of which was infected by Sendai viruses. As shown in FIG. 5A, DG75 cells transfected with LMP-1, but not EBNA-2, exhibited a marked increase in IFN-β RNA levels upon Sendai virus infection. The expression of EBNA-2 was confirmed by western blot (data not shown). These results indicate that LMP-1, but not EBNA2, can prime type III latency cells for the expression of IFNs.

LMP-1 mediated priming is similar to IFN priming, as demonstrated by FIG. 5B. However, due to the fact that LMP-1 did not induce IFN in DG75 cells in our system (70), the priming effect may be due to gene(s) regulated by LMP-1.

### Example 5

The following example demonstrates that signaling derived from LMP-1 is required for priming cells for the expression of IFNs.

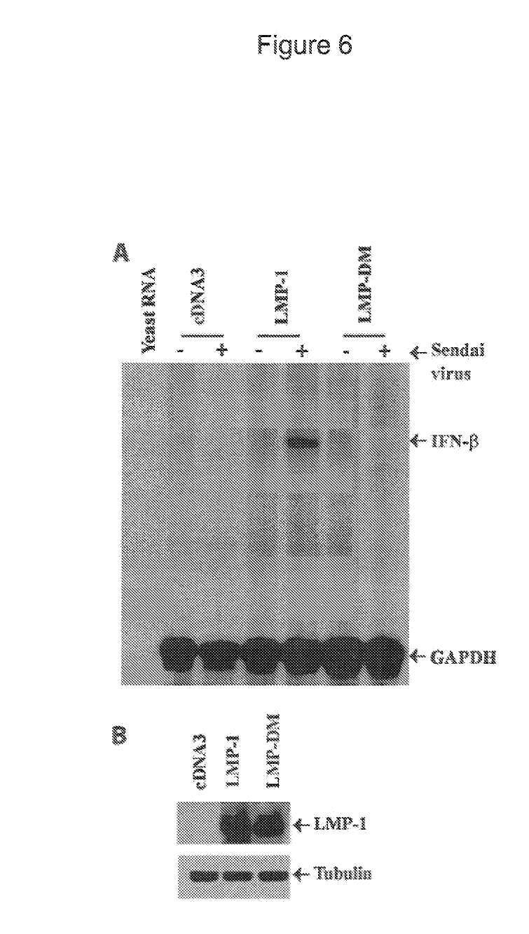

LMP-1 is an integral membrane protein with two regions in the C terminus (CTARs) that have been shown to initiate signaling processes such as the activation of NF-κB and IRF-7. LMP-DM contains mutations in both CTARs of LMP-1 and fails to activate NF-κB and IRF-7 (79). LMP-1 or LMP-DM and a CD4-expression plasmid were transfected into dG75 cells and the priming effect of LMP-1 was examined. As shown in FIG. 6A, while Sendai virus infection of LMP-1 transfected DG75 cells caused a marked increase in IFN-β RNA, LMP-DM seemed to have no effect. The expression of LMP-1 proteins was confirmed and is demonstrated in FIG. 6B. Thus, signaling from LMP-1 CTARs is required for the priming action.

### Example 6

The following example demonstrates that NF-κB contributes to LMP-1-mediated priming in DG75 cells.

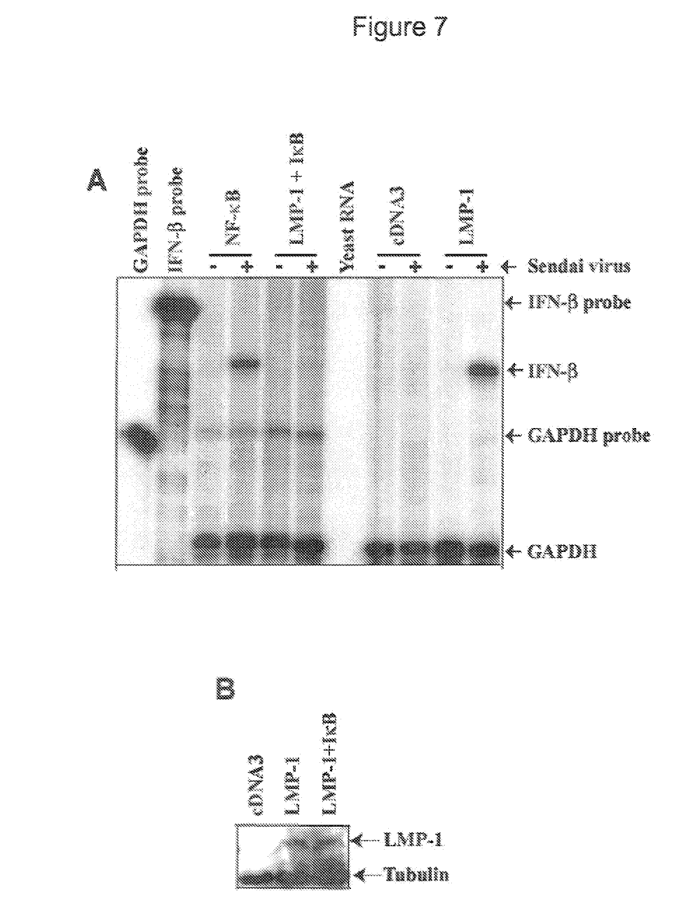

Because both LMP-1 CTARs can activate NF-κB, and since NF-κB plays a pivotal role in the viral latency, the involvement of NF-κB in LMP-1-mediated priming was examined. As shown in FIG. 7A, LMP-1 along primed DG75 cells for high IFN production induced by Sendai virus infection. However, in the presence of IkappaB (KκB), the priming effect of LMP-1 was abolished. IkB was also able to block LMP-1-mediated activation of NF-κB (data not shown).

Because overexpression of NF-κB can induce IRF-7 in DG75 cells, we examined whether NF-κB activation alone might contribute to the priming process of LMP-1. As shown in FIG. 7A, overexpression of NF-κB (p65+p50) was sufficient to prime cells for IFN production in DG75 cells. The activation of NF-κB by p65 and p50 was confirmed by NF-κB reporter assays (data not shown). Thus, these data suggest that NF-κB is involved in LMP-1-mediated priming action.

### Example 7

The following example demonstrates that IRF-7 may be a factor involved in the priming process of LMP-1.

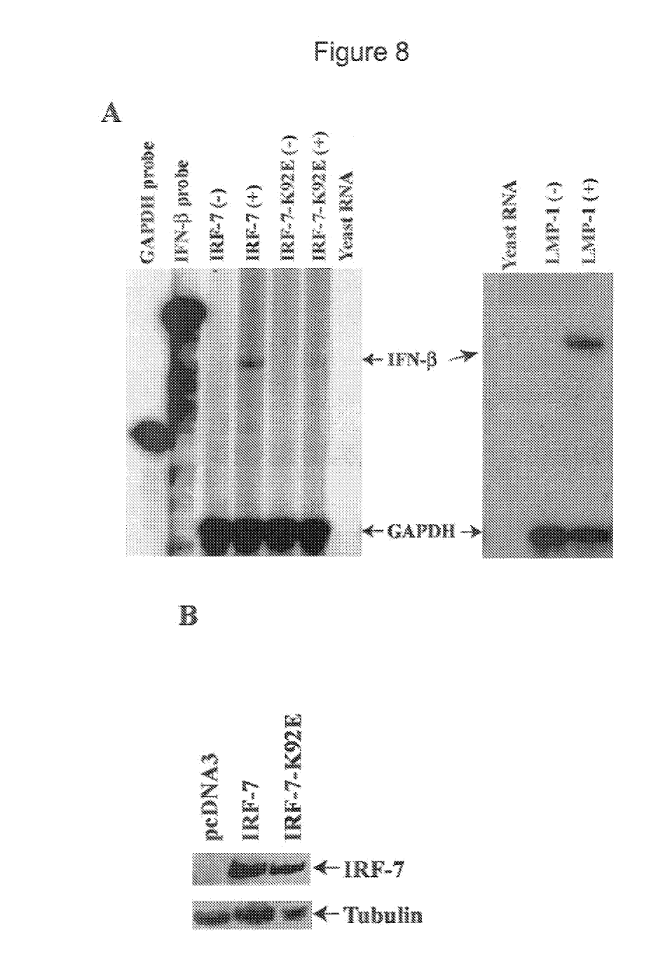

Because LMP-DM failed to induce IRF-7, and NF-κB is required for induction of IRF-7 (79), data in FIGS. 6 and 7 also suggest the potential involvement of IRF-7 in the priming process of LMP-1. In addition, the essential role of IRF-7 in type I IFN production has been well established (4, 32, 41, 68). IRF-7 and its DNA-binding mutant (IRF-7K92E) were used for transfection and their roles in LMP-1-mediated priming action were examined. As shown in FIG. 8, while wild type IRF-7 was capable of priming the transfected cells, IRF-7 DNA binding mutant (IRF-7K92E) failed to prime the cells. These data suggest that induction of IRF-7 by LMP-1 may be an important step in priming cells for IFN production.

### Example 8

The following example demonstrates that EBV-transformed cells, from where the personalized IFNs are derived, and other commercial sources for IFN productions (Namalwa and fresh isolated PBMCs) produce similar levels of IFNs upon induction by viral infection.

Namalwa is a human Burkitt's lymphoma cell line that has been used to produce commercial IFNs (e.g., Welferon from GlaxoSmithKline and Sumeriferon from Sumitomo Pharmaceuticals Co.). In addition, natural IFNs are also produced from freshly isolated PBMCs (e.g., Multiferon from Viragen) that have not been superinfected. IB4 is an EBV-Transformed cell line and was used for the comparative study as the cell line is considered a prototypical EBV-transformed cells in vitro. (9, 10, 12, 20, 30, 33)

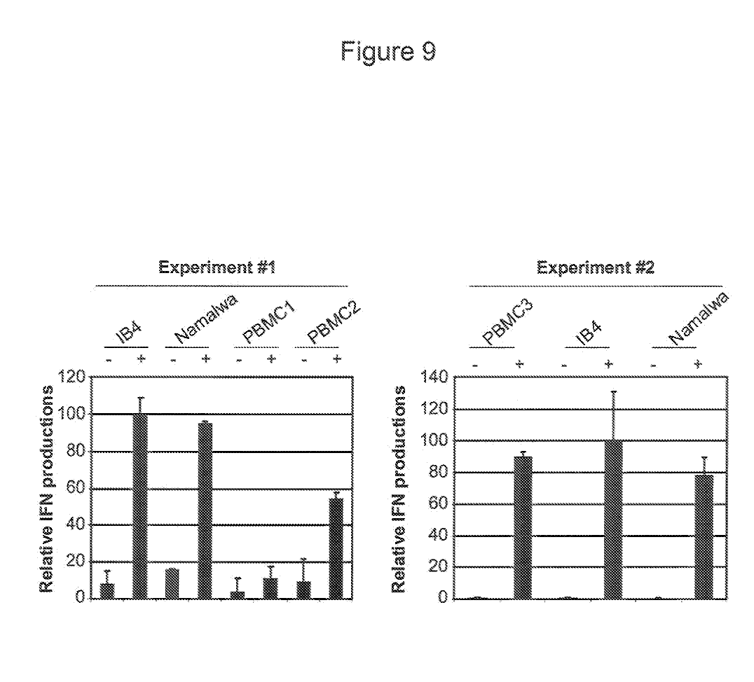

IB4, Namalwa, and freshly isolated PBMCs from three individuals were infected using equal amounts of Sendai viruses from the same batch as described in the examples above, and IFN production was monitored by ELISA. As shown in FIG. 9, comparable amounts of IFNs were produced from IB4 and Namalwa cell lines. The production of IFNs from fresh PBMCs is varied among individuals. In FIG. 9, the “+” and “−” symbols indicate with or without Sendai virus infection, respectively. The IFN productions from these cells were not primed by IFN or any other agents.

The abbreviations used above include: EBV, Epstein-Barr virus; LMP-1, latent membrane protein 1; HA, hemagglutinin; IFN, inteferon; IRF-7, IFN regulator factor 7; NF-κB, nuclear factor kappaB; IκB, inhibitor kappaB; ISG, IFN stimulated gene; FBS, fetal bovine serum; DMEM, Dulbecco's modified Eagle's Medium; PBS, phosphate buffered saline.

Each publication or reference cited herein is incorporated herein by reference in its entirety.

### REFERENCES

1. Abbot, S. D., M. Rowe, K. Cadwallader, A. Ricksten, J. Gordon, F. Wang, L. Rymo, and A. B. Rickinson. 1990. Epstein-Barr virus nuclear antigen 2 induces expression of the virus-encoded latent membrane protein. J Virol 64:2126-34.
2. Adolf, G. R., O. A. Haas, P. Fischer, and P. Swetly. 1982. Spontaneous production of alpha- and beta-interferon in human lymphoblastoid and lymphoma cell lines. Arch Virol 72:169-78.
3. Aman P., and A. von Gabain. 1990. An Epstein-Barr virus immortalization associated gene segment interferes specifically with the IFN-induced anti-proliferative response in human B-lymphoid cell lines. EMBO J 9:147-52.
4. Au, W. C., P. A. Moore, D. W. LaFleur, B. Tombal, and P. M. Pitha. 1998. Characterization of the interferon regulatory factor-7 and its potential role in the transcription activation of interferon A genes. J Biol Chem 273:29210-7.
5. Ben-Bassat, H., N. Goldblum, S. Mitrani, T. Goldblum, J. M. Yoffey, M. M. Cohen, Z. Bentwith, B. Ramot, E. Klein, and G. Klein. 1977. Establishment in continuous culture of a new type of lymphocyte from a “burkitt-like” malignant lymphoma (line D.G.-75). Int. J. Cancer 19:27-33.
6. Boumpas, D. T., J. J. Hooks, M. Popovic, G. C. Tsokos, and D. L. Mann. 1985. Human T-cell leukemia/lymphoma virus I and/or Epstein-Barr virus-infected B-cell lines spontaneously produce acid-labile alpha-interferon. J Clin Immunol 5:340-4.
7. Browne, E. P., B. Wing, D. Coleman, and T. Shenk. 2001. Altered cellular mRNA levels in human cytomegalovirus-infected fibroblasts: viral block to the accumulation of antiviral mRNAs. J Virol 75:12319-30.
8. Burysek, L., and P. M. Pitha. 2001. Latently expressed human herpesvirus 8-encoded interferon regulatory factor 2 inhibits double-stranded RNA-activated protein kinase. J Virol 75:2345-52.
9. Cahir-McFarland, E. D., D. M. Davidson, S. L. Schauer, J. Duong, and E. Kieff. 2000. NF-kappa B inhibition causes spontaneous apoptosis in Epstein-Barr virus-transformed lymphoblastoid cells. Proc Natl Acad Sci USA 97:6055-60.
10. Cahir McFarland, E. D., K. M. Izumi, and G. Mosialos. 1999. Epstein-barr virus transformation: involvement of latent membrane protein 1-mediated activation of NF-kappaB. Oncogene 18:6959-64.
11. Calender, A., M. Billaud, J. P. Aubry, J. Bauchereau, M. Vuillaume, and G. M. Lenoir. 1987. Epstein-Barr virus (EBV) includes expression of B-cell activation markers on in vitro infection of EBV-negative B-lymphoma cells. Proc Natl Acad Sci USA 84:8060-8064.
12. Carter, K. L., E. Cahir-McFarland, and E. Kieff 2002. Epstein-barr virus-induced changes in B-lymphocyte gene expression. J Virol 76:10427-36.
13. Cassady, K. A. 2005. Human cytomegalovirus TRS1 and IRS1 gene products block the double-stranded-RNA-activated host protein shutoff response induced by herpes simplex virus type I infection. J Virol 79:8707-15.
14. Chen, H., L. Hutt-Fletcher, L. Cao, and S. D. Hayward. 2003. A positive autoregulatory loop of LMP1 expression and STAT activation in epithelial cells latently infected with Epstein-Barr virus. J Virol 77:4139-48.
15. Chou, J., and B. Roizman, 1992. The gamma 1(34.5) gene of herpes simplex virus 1 precludes neuroblastoma cells from triggering total shutoff protein synthesis characteristic of programed cell death in neuronal cells. Proc Natl Acad Sci USA 89:3266-70.
16. Clarke, P. A., N. A. Sharp, J. R. Arrand, and M. J. Clemens. 1990. Epstein-Barr virus gene expression in interferon-treated cells. Implications for the regulation of protein synthesis and the antiviral state. Biochim Biophys Acta 1050:167-73.
17. Deonarain, R., A. Aleami, M. Alexiou, M. J. Dallman, D. R. Gewert, and A. C. Porter. 2000. Impaired antiviral response and alpha/beta interferon induction in mice lacking beta interferon. J Virol 74:3404-9.
18. Enoch, T., K. Zinn, and T. Maniatis. 1986. Activation of the human beta-interferon gene requires an interferon-inducible factor. Mol Cell Biol 6:801-10.
19. Fries, K. L., W. E. Miller, and N. Raab-Traub. 1996. Epstein-Barr virus latent membrane protein 1 blocks p53-mediated apoptosis through the induction of the A20 gene. J Virol 70:8653-8659.
20. Frost, V. S. Delikat, S. Al-Mehairi, and A. J. Sinclair. 2001. Regulation of p27KIP1 in Epstein-Barr virus-immortalized lympholastoid cell lines involves non-apoptotic caspase cleavage. J Gen Virol 82:3057-66.
21. Fujita, T., S. Saito, ad S. Kohno. 1979. Priming increases the amount of interferon mRNA in poly(rl).poly(rC)-treated L cells. J Gen Virol, 45:301-8.
22. Ghosh, D., and E. Kieff. 1990. cis-acting regulatory elements near the Epstein-Barr virus latent-infection membrane protein transcriptional start site. J Virol 64:1855-8.
23. Gires, O., U. Zimber-Strobl, R. Gonnella, M. Ueffing, G. Marschall, R. Zeidler, D. Pich, and W. Hammerschmidt. 1997. Latent membrane protein 1 of Epstein-Barr virus mimics a constitutively active receptor molecule. EMBO J 16.
24. Gotoh, B., T. Komatsu, K. Takeuchi, and J. Yokoo. 2002. Paramyxovirus strategies for evading the interferon response. Rev Med Virol 12:337-57.
25. Gotoh, B., K. Takeuchi, T. Komatsu, and J. Yokoo. 2003. The STAT2 activation process is a crucial target of Sendia virus C protein for the blockade of alpha interferon signaling. J Virol 77:3360-70.
26. Guttridge, D. C., M. W. Mayo, L. V. Madrid, C. Y. Wang, and A. S. Baldwin, Jr. 2000. NF-kappaB-induced loss of MyoD messenger RNA: possible role in muscle decay and cachexia. Science 289:2363-6.
27. Hahn, A. M., L. E. Huye, S. Ning, J. Webster-Cyriaque and J. S. Pagano, 2005. Interferon regulatory factor 7 is negatively regulated by the Epstein-Barr virus immediate-early gene, BZLF-1. J Virol 79:10040-52.
28. Havell, E. A., and J. Vilcek. 1972. Production of high-titered interferon in cultures of human diploid cells. Antimicrob Agents Chemother 2:476-84.
29. He, B., M. Gross, and B. Roizman. 1997. The gamma(1)34.5 protein of herpes simplex virus 1 complexes with protein phosphatase alpha to dephosphorylate the alpha subunit of the eukaryotic translation initiation factor 2 and preclude the shutoff of protein synthesis by double-stranded RNA-activated protein kinase. Proc Natl Acad Sci USA 94:843-8.
30. Henderson, A., S. Ripley, M. Heller, and E. Kieff. 1983. Chromosome site for Epstein-Barr virus DNA in a Burkitt tumor cell line and in lymphocytes growth-transformed in vitro. Proc. Natl. Acad. Sci. USA 80:1987-1991.
31. Higuchi, M., E. Kieff, and K. M. Izumi. 2002. The Epstein-Barr virus latent membrane protein 1 putative Janus kinase 3 (JAK3) binding domain does not mediate JAK3 association or activation in B-lymphoma or lymphoblastoid cell lines. J Virol 76:455-9.
32. Honda, K, H. Yanai, H. Negishi, M. Asagiri, M. Sato, T. Mizutani, N., Shimada, Y. Ohba, A. Takaoka, N. Yoshida, and T. Taniguchi. 2005. IRF-7 is the master regulator of type-I interferon-dependent immune responses. Nature 434:772-7.
33. Hurley, E. A., L. D. Klaman, S. Agger, J. B. Lawrence, and D. A. Thorley-Lawson. 1991. The prototypical Epstein-Barr virus-transformed lymphoblastoid cell line IB4 is an unusual variant containing integrated but no episomal viral DNA. J Virol 65:3958-63.
34. Kieff, E. 1996. Epstein-Barr virus and its replication, p. 2343-2396. In B. N. Fields, D. M. Knipe, and P. M. Howley (ed.), Virology, 3rd Edition, Lippinscott-Raven Publishers, Philadelphia, Pa.
35. Komatsu, T., K. Takeuchi, J. Yokoo, and B. Gotoh. 2002. Sendi virus C protein impairs both phosphorylation and dephosphorylation processes of Statl. FEBS Lett 511:139-44.
36. Komatsu, T., K. Takeuchi, J. Yokoo, Y. Tanaka, and B. Gotoh. 2000. Sendai virus blocks alpha interferon signaling to signal transducers and activators of transcription. J Virol 74:2477-80.
37. Krug, L. T., V. P. Pozharskaya, Y. Yu, N. Inoue, and M. K. Offermann. 2004. Inhibition of infection and replication of human herpesvirus 8 in microvascular endothelial cells by alpha interferon and phosphonoformic acid. J Virol 78:8359-71.
38. Levy, D. E., I. Marie, E. Smith, and A. Prakash. 2002. Enhancement and diversification of IFN induction by IRF-7-mediated positive feedback. J Interferon Cytokine Res 22:87-93.
39. Liebowitz, D., D. Wang and E. Kieff. 1986. Orientation and patching of the latent infection membrane protein encoded by Epstein-Barr virus J. Virol 58:233-7.
40. Lin, R., Y. Mamane, and J. Hiscott. 2000. Multiple regulatory domains control IRF-7 activity in response to virus infection. J Biol Chem. 275:34320-7.
41. Marie, I., J. E. Durbin, and D. E. Levy. 1998. Differential viral induction of distinctinterferon-alpha genes by positive feedback through interferon regulatory factor-7. EMBO J 17:6660-9.
42. Miller, W. E., H. S. Earp, and N. Raab-Traub. 1995. The Epstein-Barr Virus Latent Membrane Protein1 Induces Expression of the Epidermal Growth Factor Receptor. J Virol. 69:4390-4398.
43. Mittnacht, S., P. Straub, H. Kirchner, and H. Jacobsen 1988. Interferon treatment inhibits onset of herpes simplex virus immediate-early transcription. Virology 164:201-10.
44. Monini, P., F. Carlini, M. Sturzl, P. Rimessi, F. Superti, M. Franco, G. Melucci-Vigo, A. Cafaro, D. Goletti, C. Sgadari, S. Butto, P. Leone, C. Chiozzani, C. Barresi, A. Tinari, A. Bonaccorsi, M. R. Capobianchi, M. Giuliani, A. di Carlo, M. Andreoni, G. Rezza, and B. Ensoli. 1999. Alpha interferon inhibits human herpesvirus 8 (HHV-8) reactivation in primary effusion lymphoma cells and reduces HHV-8 load in cultured peripheral blood mononuclear cells. J Virol. 73:4029-41.
45. Morrison, T. E., A. Mauser, A. Wong, J. P. Ting, and S. C. Kenney. 2001. Inhibition of IFN-gamma signaling by an Epstein-Barr virus immediate-early protein. Immunity 15:787-99.
46. Mossman, K. L., and J. R. Smiley. 2002. Herpes simplex virus ICP0 and ICP34.5 counteract distinct interferon-induced barriers to virus replications. J Virol 76:1995-8.
47. Nir, U., B. Cohen, L. Chen, and M. Revel. 1984. A human IFN-beta 1 gene deleted of promoter sequences upstream from the TATA box is controlled post-transcriptionally by dsRNA. Nucleic Acids Res 12:6979-93.
48. Nonkwelo, C., J. Skinner, A. Bell, A. Rickinson, and J. Sample 1996. Transcription start sites downstream of the Epstein-Barr virus (EBV) Fp promoter in early-passage Burkitt lymphoma cells define a fourth promoter for expression of the EBV EBNA-1 protein. J Virol 70:623-627.
49. Oberman, F., and A. Panet. 1989 Characterization of the early steps of herpes simplex virus replication in interferon-treated human cells. J Interferon Res 9:563-71.
50. Oberman, F., and A. Panet. 1988. Inhibition of transcription of herpes simplex virus immediate early genes in interferon-treated human cells. J Gen Virol 69 (Pt 6):1167-77.
51. Peters, K. L., H. L. Smith, G. R. Stark, and G. C. Sen. 2002. IRF-3-dependent, NFkappa B- and JNK-independent activation of the 561 and IFN-beta genes in response to double-stranded RNA. Proc Natl Acad Sci USA 99:6322-7.
52. Pozharskaya, V. P., L. L. Weakland, and M. K. Offermann. 2004. Inhibition of infectious human herpesvirus 8 production by gamma interferon and alpha interferon in BCBL-1 cells. J Gen Virol 85:2779-87.
53. Richardson, C., C. Fielding, M. Rowe, and P. Brennan. 2003. Estein-Barr virus regulates STAT1 through latent membrane protein 1. J Virol 77:4439-43.
54. Rickinson, A. B., and E. Kieff. 1996. Epstein-Barr Virus, p. 2397-2446. In B. N. Fields, D. M. Knipe, and P. M. Howley (ed.), Virology, 3rd Edition. Lippinscott-Raven Publishers, Philadelphia, Pa.
55. Roberts, R. M., L. Liu, Q. Guo, D. Leaman, and J. Bixby. 1998. The evolution of the type I interferons. J Interferon Cytokine Res 18:805-16.
56. Samuel, C. E. 2001. Antiviral actions of interferons. Clin Microbiol Rev 144:778-809, table of contents.
57. Sen, G. 2001, Viruses and Interferons. Annu. Rev. Microbiol. 55:255-81.
58. Sharp, N. A., J. R. Arrand, and M. J. Clemens. 1989. Epstein-Barr virus replication in interferon-treated cells. J Gen Virol 70 (Pt 9):2521-6.
59. Sharp, T. V., M. Schwemmle, I. Jeffrey, K. Laing, H. Mellor, C. G. Proud, K. Hilse, and M. J. Clemens. 1993. Comparative analysis of the regulation of the interferon-inducible protein kinase PKR by Epstein-Barr virus RNAs EBER-1 and EBER-2 and adenovirus VAI RNA. Nucleic Acids Res 21:4483-90.
60. Simmen, K. A., J. Singh, B. G. Luukkonen, M. Lopper, A. Bittner, N. E. Miller, M. R. Jackson, T. Compton, and K. Fruh. 2001. Global modulation of cellular transcription by human cytomegalovirus is initiated by glyoprotein B. Proc Natl Acad Sci USA 98:7140-5.
61. Stewart, W. E., 2nd, L. B. Gosser, and R. Z. Lockart, Jr. 1971. Priming: a nonantiviral function of interferon. J Virol 7:792-801.
62. Tolo, Y, H. L. Kauppinen, G. Alm, A. Peters, E. Lindeberg, V. Wahlstedt-Froberg, and J. Parkkinen. 2001. Development of a highly purified multicomponent leukocyte IFN-alpha product. J. Interferon Cytokine Res 21:913-20.
63. Tsang, S. F., F. Wang, K. M. Izumi, and E. Kieff. 1991. Delineation of the cis-acting element mediating EBNA-2 transactivation of latent infection membrane protein expression. J Virol 65:6765-71.
64. Wang, C. Y., D. C. Guttridge, M. W. Mayo, and A. S. Baldwin, Jr. 1999. NF-kappaB induces expression of the Bcl-2 homologue A1/Bfl-1 to preferentially suppress chemotherapy-induced apotosis. Mol Cell Biol 19:5923-9.
65. Wang, D., D. Leibowitz, and E. Kieff. 1985. An EBV membrane protein expressed in immortalized lymphocytes transforms established rodent cells. Cell 43:831-840
66. Wang F., C. Gregory, C. Sample, M. Rowe, D. Liebowitz, R. Murray, A. Rickinson, and E. Kieff. 1990. Epstein-Barr virus latent membrane protein (LMP1) and nuclear proteins 2 and 3C are effectors of phenotypic changes in B lymphocytes EBNA-2 and LMP1 cooperatively induce CD23. J Virol 64:2309-18.
67. Wang, F., S. F. Tsang, M. G. Kurilla, J. I. Cohen, and E. Kieff. 1990. Epstein-Barr virus nuclear antigen 2 transactivates latent membrane protein LMP1. J Virol 64:3407-3416.
68. Wathelet, M. G., C. H. Lin, B. S. Parekh, L. V. Ronco, P. M. Howley, and T. Maniatis. 1998. Virus infection induces the assembly of coordinately activated transcription factors or the IFN-beta enhacer in vivo. Mol Cell 1:507-518.
69. Yoshizaki, T., H. Sato, M. Furukawa, and J. S. Pagano. 1998. The expression of matrix metalloproteinase 9 is enhanced by Epstein-Barr virus latent membrane protein 1. Proc Natl Acad Sci USA 95:3621-3626.
70. Zhang, J., S. C. Das, C. Kotalik, A. K Pattnaik, and L. Zhang. 2004. The Latent Membrane Protein 1 of Epstein-Barr Virus Establishes an Antivrial State via Induction of Interferon-stimulated Genes. J Biol Chem 279:46335-42.
71. Zhang, J., J. Wang, C. Wood, D. Xu, and L. Zhang. 2005. Kaposi's Sarcoma-Associated Herpesvirus/Human Herpesvirus 8 Replication and Transcription Activator Regulates Viral and Cellular Genes via Interferon-Stimulated Response Elements. J. Virol. 79:5640-5652.
72. Zhang, L., 2000. Gradient temperature hybridization using a thermocycler for RNase protection assays. Molecular biotechology 14:73-75.
73. Zhang, L., K. Hong, J. Zhang, and J. Pagano. 2004. Multiple signal transducers and activators of transcription (STATs) are induced by EBV LMP-1. Virology 323:141-152.
74. Zhang, L., and J. S. Pagano. 2000. Interferon regulators factor 7 is induced by Epstein-Barr virus latent membrane protein 1. J. Virol. 74:1061-8.
75. Zhang, L., and J. S. Pagano. 2001. Interferon Regulatory Factor 7 mediates the activation of Tap-2 by Epstein-Barr virus latent membrane protein 1. J Virol 75:341-50.
76. Zhang, L., and J. S. Pagano. 2001. Interferon Regulatory Factor 7: a Key Cellular Mediator of LMP-1 in EBV Latency and Transformation. Seminars in Cancer Biology 11:445-53.
77. Zhang, L., and J. S. Pagano. 1997. IRF-7, a new interferon regulator factor associated with Epstein-Barr virus latency. Mol Cell Biol 17:5748-5757.
78. Zhang, L., and J. S. Pagano. 2002. Review: Structure and Function of IRF-7. J Interferon Cytokine Res 22:95-101.
79. Zhang, L., L. H. Wu, K. Hong, and J. S. Pagano. 2001. Intracellular signaling molecules activated by Epstein Barr viruses for induction of interferon regulatory factor 7. J. Virol. 75:12393-401.
80. Zhang, L., J. Zhang, Q. Lambert, C. J. Der, L. Del Valle, J. Miklossy, K. Khalili, Y. Zhou, and J. S. Pagano. 2004. Interferon regulatory factor 7 is associated with Epstein-Barr virus-transformed central nervous system lyphoma and has onogenic properties. J Virol 78:12987-95.
80. Zhu, H., J. P. Cong, and T. Shenk. 1997. Use of differential display analysis to assess the effect of human cytomegalovirus infection on the accumulation of cellular RNAs: induction of interferon-responsive RNAs. Proc Natl Acad Sci USA 94:13985-90.

While various embodiments of the present invention have been described in detail, it is apparent that modifications and adaptations of those embodiments will occur to those skilled in the art. It is to be expressly understood, however that such modifications and adaptations are within the scope of the present invention, as set forth in the following exemplary claims.

What is claimed is:

1. A method to produce interferon (IFN), comprising:
a) providing a cell that as been primed for interferon production by infection of the cell with a herpesvirus or transfection of the cell with a portion of a herpesvirus genome;
b) infecting the cell with a virus or providing the cell with a virus-like stimulus to induce the cell to produce IFN; and
c) recovering the IFN from the cell.

2. The method of claim 1, wherein the cell is a human cell.

3. The method of claim 2, wherein the human cell is a peripheral blood mononuclear cell.

4. The method of claim 2, wherein the human cell is a lymphocyte.

5. The method of claim 2, wherein the human cell is a B lymphocyte.

6. The method of claim 1, wherein the herpesvirus is a gamma herpesvirus.

7. The method of claim 6, wherein the gamma herpesvirus is an Epstein-Barr virus (EBV).

8. The method of claim 1, wherein the portion of the herpesvirus genome comprises a nucleic acid molecule encoding LMP-1.

9. The method of claim 8, wherein the portion of the herpesvirus genome further comprises at least one nucleic acid molecule encoding a protein selected from: EBNA-1, EBNA-2, EBNA-3A, EBNA-3B, EBNA-3C and EBNA-LP, LMP-2A and LMP-2B.

10. The method of claim 1, wherein the portion of the herpesvirus genome comprises a nucleic acid molecule encoding at least one CTAR domain of LMP-1.

11. The method of claim 1, wherein the virus is selected from the group consisting of: Sendai virus, Newcastle disease virus and vesicular stomatitis virus.

12. The method of claim 1, wherein the virus is Sendai virus.

13. The method of claim 1, wherein the virus-like stimulus is double-stranded RNA.

14. The method of claim 1, wherein step (c) of recovering comprises isolating IFN from the cell or a lysate thereof.

15. The method of claim 1, wherein step (c) of recovering comprises purifying IFN from the cell or a lysate thereof.

16. The method of claim 1, wherein the IFN is a type I interferon.

17. The method of claim 1, wherein the IFN is interferon-α or a species thereof.

18. The method of claim 1, wherein the IFN is interferon-β or a species thereof.

19. The method of claim 1, wherein the cell is isolated from a human subject.

20. The method of claim 19, wherein step (c) of recovering comprises purifying IFN from the cell or a lysate thereof.

21. The method of claim 20, further comprising a step of administering the purified IFN to the human subject from which the cell was isolated.

22. The method of claim 21, wherein the subject has a disease or condition selected from the group consisting of: Hepatitis C virus (HCV) infection, Hepatitis B virus (HBV) infection, cancer, and multiple sclerosis (MS).

23. Isolated or purified IFN produced by the method of claim 1.

24. A method to produce interferon (IFN), comprising:
a) providing a cell that expresses LMP-1 or a portion thereof that contains at least one CTAR domain;
b) infecting the cell with a virus or providing the cell with a virus-like stimulus to induce the cell to produce IFN; and
c) recovering the IFN from the cell.

25. The method of claim 24, wherein the cell is a human cell.

26. The method of claim 25, wherein the human cell is a peripheral blood mononuclear cell.

27. The method of claim 25, wherein the human cell is a lymphocyte.

28. The method of claim 25, wherein the human cell is a B lymphocyte.

29. The method of claim 24, wherein the cell comprises an expression vector comprising a nucleic acid molecule encoding LMP-1 or a portion thereof that contains at least one CTAR domain.

30. The method of claim 24, wherein the virus is selected from the group consisting of: Sendai virus, Newcastle disease virus and vesicular stomatitis virus.

31. The method of claim 24, wherein the virus is Sendai virus.

32. The method of claim 24, wherein the virus-like stimulus is double-stranded RNA.

33. The method of claim 24, wherein step (c) of recovering comprises isolating IFN from the cell or a lysate thereof.

34. The method of claim 24, wherein step (c) of recovering comprises purifying IFN from the cell or a lysate thereof.

35. The method of claim 24, wherein the IFN is a type I interferon.

36. The method of claim 24, wherein the IFN is interferon-α or a species thereof.

37. The method of claim 24, wherein the IFN is interferon-β or a species thereof.

38. Isolated or purified IFN produced by the method of claim 24.

39. A method to produce personalized interferon (IFN), comprising:
a) obtaining a cell from a subject;
b) priming the cell for interferon production by infection of the cell with a herpesvirus or transfection of the cell with a portion of a herpesvirus genome;
c) infecting the cell with a virus to induce the cell to produce IFN; and
d) recovering the IFN from the cell.

40. Isolated or purified IFN produced by the method of claim 39.

41. A method to produce personalized interferon (IFN), comprising:
a) obtaining a cell from a subject;
b) transfecting LMP-1 or a portion thereof that contains at least one CTAR domain into the cell;
c) infecting the cell with a virus to induce the cell to produce IFN; and
d) recovering the IFN from the cell.

42. Isolated or purified IFN produced by the method of claim 41.

43. A method to treat a patient with a disease or condition capable of being treated by interferon (IFN) therapy, comprising:
a) obtaining a cell from the patient;
b) priming the cell for interferon production by infection of the cell with a herpesvirus or transfection of the cell with a portion of a herpesvirus genome;
c) infecting the cell with a virus to induce the cell to produce IFN;
d) isolating the IFN from the cell; and
e) administering the isolated IFN to the patient.

44. The method of claim 43, wherein the patient has a disease or condition selected from the group consisting of: Hepatitis C virus (HCV) infection, Hepatitis B virus (HBV) infection, cancer, and multiple sclerosis (MS).

45. The method of claim 43, wherein the patient has hepatitis.

46. The method of claim 43, wherein the cell is a B lymphocyte.

47. The method of claim 43, wherein the portion of the herpesvirus genome comprises a nucleic acid molecule encoding LMP-1 or a portion thereof that contains at least one CTAR domain.

48. The method of claim 43, wherein the herpesvirus is EBV.

49. The method of claim 43, wherein the virus is selected from the group consisting of: Sendai virus, Newcastle disease virus and vesicular stomatitis virus.

50. The method of claim 49, wherein the virus is Sendai virus.

51. The method of claim 43, wherein the step of isolating comprises purifying the IFN.

52. A method to treat a patient with a disease or condition treatable with interferon (IFN) therapy, comprising administering isolated IFN to the patient, wherein the isolated IFN has been produced by the process comprising:
a) infecting an herpesvirus-immortalized type III latency cell with a virus to induce the cell to produce IFN; and
b) isolating the IFN from the cell.

53. The method of claim 52, wherein the patient has a disease or condition selected from the group consisting of: Hepatitis C virus (HCV) infection, Hepatitis B virus (HBV) infection, cancer, and multiple sclerosis (MS).

54. The method of claim 52, wherein the patient has hepatitis.

55. The method of claim 52, wherein the herpesvirus is EBV.

56. The method of claim 52, wherein the virus is selected from the group consisting of: Sendai virus, Newcastle disease virus and vesicular stomatitis virus.

57. The method of claim 56, wherein the virus is Sendai virus.

58. The method of claim 52, wherein the step of isolating comprises purifying the IFN.


SEQUENCE LISTING (1 sequence)

SEQ ID NO: 1 - protein, 386 aa, Epstein Barr Virus
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD